(19) United States
(12) Patent Application Publication
(10) Pub. No.: US 2026/0066126 A1
(43) Pub. Date: Mar. 5, 2026

(54) SYSTEM AND METHOD FOR HEALTHCARE DIAGNOSTICS USING FOUNDATION MODELS WITH UNCERTAINTY TRIAGE

(71) Applicants: Arvind Kumar CHAUDHARY, Lansdale, PA (US); Akshar PATEL, Phoenix, AZ (US); Ronish B. PATEL, Townsend, DE (US); Mourad BOUTAHIR, Meknes (MA)

(72) Inventors: Arvind Kumar CHAUDHARY, Lansdale, PA (US); Akshar PATEL, Phoenix, AZ (US); Ronish B. PATEL, Townsend, DE (US); Mourad BOUTAHIR, Meknes (MA)

(21) Appl. No.: 19/380,801

(22) Filed: Nov. 5, 2025

(51) Int. Cl. G16H 50/20; G06N 3/047; G16H 10/60; G16H 30/40; G16H 50/70

(52) U.S. Cl. CPC G16H 50/20; G06N 3/047; G16H 10/60; G16H 30/40; G16H 50/70

(57) ABSTRACT

The present invention discloses a system and method for healthcare diagnostics using foundation models with uncertainty triage, designed to deliver reliable, explainable, and safety-assured diagnostic outcomes across multimodal clinical data. The invention integrates a foundation model processor pretrained on diverse medical datasets with an uncertainty estimation processor configured to quantify epistemic and aleatoric uncertainties in diagnostic predictions. A triage control unit dynamically classifies cases into high, medium, and low-confidence categories based on computed uncertainty indices, ensuring that only high-confidence cases are automatically finalized, while uncertain or ambiguous cases are routed for clinician review. The system further incorporates a feedback adaptation processor that recalibrates model parameters and uncertainty thresholds based on expert feedback, maintaining alignment with clinical reliability standards over time. Implemented as a hardware-integrated diagnostic device, the invention supports real-time inference, secure data handling, and interpretability visualization through uncertainty heatmaps and attention overlays.

### TECHNICAL FIELD

The present invention pertains to the field of artificial intelligence-assisted healthcare diagnostics. More particularly, it relates to systems and methods for performing diagnostic predictions using large-scale multimodal foundation models in conjunction with an uncertainty triage subsystem that evaluates model confidence levels, stratifies diagnostic outcomes, and dynamically assigns diagnostic routes to automated or clinician-involved decision pathways.

### BACKGROUND OF THE INVENTION

Conventional AI-driven diagnostic systems often rely on deep learning architectures trained on limited, domain-specific datasets. While these models demonstrate high performance under ideal conditions, they typically fail to generalize to unseen cases, exhibit overconfidence in uncertain predictions, and lack a mechanism to assess or communicate uncertainty. This overconfidence poses substantial risks in clinical practice where misclassification may lead to patient harm.

Recent advancements in foundation models, such as large multimodal transformers trained across diverse medical and non-medical datasets, have introduced generalizable representations that can perform diagnostic tasks across modalities. However, these models still lack inherent mechanisms to quantify predictive uncertainty and triage cases accordingly. Therefore, there exists a need for a unified system that integrates foundation models with an uncertainty-aware triage mechanism capable of ensuring reliable and interpretable diagnostic decision-making in healthcare settings.

In recent years, the field of artificial intelligence (AI) has revolutionized healthcare diagnostics, especially through the adoption of deep learning models that assist clinicians in interpreting medical images, analyzing biosignals, and predicting disease outcomes from complex datasets. These systems have demonstrated remarkable accuracy in specialized tasks such as tumor detection in radiology, diabetic retinopathy screening in ophthalmology, and cardiac arrhythmia classification from electrocardiograms (ECGs). However, most of these systems rely on task-specific neural networks trained on relatively limited and homogeneous datasets. Consequently, while they may achieve excellent performance in controlled conditions, they frequently exhibit performance degradation when applied to diverse real-world scenarios. The core problem arises from their lack of generalization, inability to quantify prediction confidence, and the absence of an adaptive decision-making layer that can handle diagnostic uncertainty. In clinical environments where the cost of misdiagnosis is high, overconfident but incorrect AI predictions can lead to severe medical errors. Thus, there is a growing need for diagnostic AI systems that not only perform accurate predictions but also provide a measure of reliability or uncertainty for each output, enabling safe integration into clinical workflows.

Traditional AI systems in healthcare are predominantly based on convolutional neural networks (CNNs) or recurrent neural networks (RNNs) designed for specific modalities, such as medical imaging or sequential patient records. For example, CNN-based architectures like ResNet, DenseNet, and U-Net have achieved significant milestones in radiology tasks, such as lesion detection or segmentation in computed tomography (CT) and magnetic resonance imaging (MRI). Similarly, RNN-based models and transformer variants have been deployed for sequential data interpretation, including patient vital sign forecasting or clinical text summarization. However, despite these advancements, the models typically operate as black boxes. They produce deterministic outputs without conveying how confident the system is about its decision. This lack of interpretability and uncertainty awareness limits their acceptance among clinicians, who require explanations and confidence estimates before trusting AI-assisted decisions in critical diagnostic settings.

Furthermore, most diagnostic AI models are developed in isolated contexts, focusing on a single data modality. For instance, a chest X-ray classifier might operate independently of patient history or laboratory results, leading to incomplete diagnostic reasoning. Healthcare diagnosis, however, is inherently multimodal, requiring the integration of image features, clinical notes, genomics data, and physiological measurements. The inability of traditional models to capture and fuse such heterogeneous data sources restricts their utility in real-world diagnosis. Although some multimodal AI architectures have been proposed, such as joint image-text transformers, they still lack robust mechanisms to assess uncertainty or adaptively route cases based on the reliability of predictions. Without uncertainty quantification, even the most advanced multimodal systems can propagate errors, making them unsafe for unsupervised clinical use.

The emergence of foundation models—large-scale neural networks pretrained on vast and diverse datasets—has introduced a new paradigm in AI for healthcare. Models like CLIP, BioGPT, and Med-PaLM have shown that a single pretrained architecture can generalize across multiple medical tasks, including visual recognition, text comprehension, and multimodal reasoning. These foundation models leverage self-supervised or contrastive learning strategies to learn generalized representations of medical knowledge, significantly reducing the data requirements for downstream tasks. Their capacity to adapt to multiple domains through fine-tuning makes them ideal candidates for diagnostic systems that must handle a wide range of inputs. However, despite their scalability and versatility, foundation models also suffer from fundamental limitations when deployed in clinical environments. Most foundation models are trained with objectives optimized for accuracy or likelihood, but not for uncertainty calibration. As a result, they tend to exhibit overconfidence, producing high-probability predictions even in ambiguous or out-of-distribution cases. In clinical diagnostics, this behavior is dangerous because the model may appear confident in a wrong decision, misleading healthcare practitioners and jeopardizing patient safety.

Another crucial drawback of existing solutions is the lack of clinical feedback integration. Once a model has been deployed, its performance in real-world environments may deviate significantly from validation results. Factors such as patient demographics, imaging protocols, and disease prevalence can shift, resulting in data distribution drift. However, current AI systems do not typically incorporate clinician feedback loops that enable recalibration based on new ground truth data. This absence of adaptive learning causes models to degrade in accuracy and reliability over time. Moreover, without active feedback-based calibration, uncertainty estimates themselves can become misaligned, leading to underestimation or overestimation of risk. An effective diagnostic system must, therefore, not only measure uncertainty but also continuously learn from clinician feedback to maintain calibration.

In summary, while AI-driven healthcare diagnostics has advanced significantly through deep learning and foundation models, critical gaps remain in uncertainty quantification, adaptive triage, feedback integration, and computational scalability. Existing models excel at deterministic classification but lack mechanisms to express confidence, distinguish between known and unknown cases, or adapt their behavior based on uncertainty. Most systems also fail to route uncertain cases for expert review or refine themselves based on human feedback. Consequently, their reliability and safety in clinical deployment remain questionable. The technical deficiencies in current solutions underscore the urgent need for a unified system that combines the scalability of foundation models with rigorous uncertainty quantification and active triage control. Such a system would bridge the gap between automated precision and human judgment, ensuring that AI becomes a trustworthy, transparent, and adaptive partner in healthcare diagnostics rather than a black-box decision-maker.

### SUMMARY OF THE INVENTION

The present invention provides an intelligent healthcare diagnostic system that integrates large-scale foundation model architectures with uncertainty-aware probabilistic inference and adaptive triage control to deliver reliable, interpretable, and safety-assured clinical decisions. The system employs a multimodal transformer-based foundation model capable of jointly analyzing heterogeneous medical data—including radiological images, textual case histories, laboratory records, and biosignal sequences—to produce contextually enriched diagnostic feature representations. A dedicated uncertainty estimation processor quantifies both epistemic and aleatoric uncertainties using Bayesian ensemble inference and stochastic sampling, thereby assigning calibrated confidence scores to each diagnostic prediction. Based on these uncertainty values, an adaptive triage control subsystem dynamically routes diagnostic cases into automated, semi-automated, or clinician-supervised decision pathways, ensuring that low-confidence or ambiguous cases are escalated for expert verification. The invention further incorporates a feedback adaptation mechanism that continuously refines model calibration using clinician-provided ground truth data, enabling self-learning and performance stabilization over time. Implemented within a hardware-integrated computing chassis equipped with probabilistic co-processors and real-time visualization units, the invention establishes a robust and interpretable diagnostic framework that harmonizes automation efficiency with clinical safety. By unifying foundation model intelligence with uncertainty quantification and adaptive triage, the system overcomes the limitations of conventional deterministic AI diagnostics, ensuring transparency, adaptability, and trustworthiness in healthcare decision-making.

The primary object of the present invention is to provide a system and method for healthcare diagnostics using foundation models integrated with uncertainty triage that ensures safe, reliable, and explainable clinical decision-making across diverse medical scenarios. The invention aims to overcome the limitations of existing AI diagnostic systems that lack uncertainty awareness, multimodal adaptability, and clinician-in-the-loop feedback. By embedding uncertainty quantification mechanisms within large-scale foundation models and coupling them with a hierarchical triage framework, the invention ensures that diagnostic decisions are dynamically adjusted according to confidence levels. This allows high-confidence cases to be processed automatically, while low-confidence or ambiguous cases are routed to expert clinicians for review, thereby minimizing diagnostic risk and improving trust in AI-assisted healthcare systems.

Another important object of the invention is to provide a multimodal diagnostic capability that integrates and harmonizes various data types—including medical imaging, clinical text, laboratory results, and biosignal data—within a unified foundation model architecture. The system leverages pretrained large-scale neural networks capable of cross-modal understanding, enabling comprehensive diagnostic reasoning that mimics human clinical judgment. This multimodal integration ensures that diagnostic conclusions are contextually aware, reducing the chances of partial or isolated interpretations that are common in single-modality systems. By embedding uncertainty estimation at each data processing level, the invention provides a confidence-weighted fusion of information across modalities, enhancing the robustness and interpretability of diagnostic outcomes.

A further object of the invention is to provide an uncertainty quantification subsystem that estimates both epistemic (model-related) and aleatoric (data-related) uncertainty during inference. The system is designed to quantify the confidence of each diagnostic prediction using Bayesian ensemble inference, Monte Carlo dropout sampling, and evidential learning techniques. This enables the diagnostic system to distinguish between confidently correct predictions, confidently incorrect predictions, and uncertain cases requiring further evaluation. Unlike existing AI systems that produce deterministic or pseudo-probabilistic confidence scores, the proposed invention generates calibrated uncertainty values that reflect the true reliability of model outputs. This object ensures that every diagnostic result is accompanied by a transparent measure of trustworthiness, enabling clinicians to make better-informed decisions.

An additional object of the invention is to provide a triage decision controller that actively manages diagnostic workflows based on the computed uncertainty levels. The controller categorizes diagnostic cases into high-confidence, moderate-confidence, and low-confidence tiers. High-confidence results are automatically finalized and reported, moderate-confidence results are subjected to ensemble re-evaluation or secondary model analysis, and low-confidence results are directed to human experts for confirmation. This triage-based routing mechanism ensures an intelligent balance between automation efficiency and diagnostic safety. The objective here is to create a self-regulating diagnostic ecosystem where automation is maximized without compromising clinical accuracy or patient safety.

Another key object of the invention is to provide a feedback adaptation subsystem that continuously refines the diagnostic model through clinician-in-the-loop interactions. The system captures expert annotations, corrections, and confirmatory diagnoses from human reviewers and uses these as feedback signals for recalibrating model uncertainty and retraining decision boundaries. This continuous feedback mechanism ensures that the system remains adaptive to new clinical contexts, evolving medical knowledge, and emerging disease patterns. Through this object, the invention establishes a dynamic calibration process that maintains diagnostic accuracy and reliability over time, even as input data distributions change.

A further object of the invention is to provide an explainable AI interface that presents diagnostic results, uncertainty visualizations, and reasoning maps in an interpretable and clinically meaningful form. The interface displays uncertainty heatmaps, attention overlays, and diagnostic rationales that allow clinicians to visually assess which regions or features influenced the model's prediction. This objective directly addresses the opacity of existing black-box AI systems by enabling traceable and interpretable diagnostic outputs, thereby fostering greater trust and accountability in AI-driven healthcare decisions.

It is also an object of the invention to provide a hardware-integrated diagnostic device capable of performing real-time uncertainty-aware inference in a clinical environment. The device is constructed as a compact, rack-mounted computational unit equipped with high-performance AI accelerators, memory units, and an uncertainty co-processor. The objective of this design is to provide a practical and deployable machine that can seamlessly integrate into hospital networks, medical imaging systems, and electronic health record (EHR) databases. Through optimized hardware architecture, the device supports low-latency inference, ensuring that uncertainty quantification and triage operations can be performed in real-time during patient evaluation.

Another object of the invention is to enable efficient computational scalability for foundation models with integrated uncertainty estimation. The system architecture is optimized to minimize the additional computational overhead introduced by probabilistic inference or ensemble methods. Through selective uncertainty sampling, parallelized inference pipelines, and model pruning strategies, the invention ensures that uncertainty quantification does not significantly compromise processing speed. The objective here is to make the uncertainty-aware diagnostic framework viable for both high-resource tertiary hospitals and lower-resource clinical centers without demanding excessive hardware capacity.

An additional object of the invention is to ensure secure and privacy-preserving data handling during diagnostic processing. The system incorporates anonymization and encryption mechanisms to ensure that patient data used for model inference or feedback remains protected in compliance with healthcare privacy regulations such as HIPAA and GDPR. The objective is to build a trusted AI diagnostic ecosystem that guarantees both clinical integrity and data security. The system further includes access control, audit trails, and tamper-proof data logging, ensuring full traceability of each diagnostic decision.

Another object of the invention is to reduce diagnostic variability across different practitioners, institutions, and equipment configurations by establishing a standardized diagnostic protocol powered by a generalizable foundation model. The uncertainty triage mechanism ensures that diagnostic outcomes remain consistent across varying patient demographics, imaging devices, and clinical workflows. This standardization objective aims to enhance equity and uniformity in healthcare diagnostics, ensuring that AI assistance delivers reliable performance across diverse populations and care settings.

Another object of the invention is to provide a fail-safe diagnostic routing mechanism that ensures that no uncertain or potentially erroneous predictions are finalized without human verification. In high-risk cases, such as cancer detection, stroke diagnosis, or cardiac anomaly classification, the system enforces strict uncertainty thresholds that automatically trigger human intervention. This object guarantees that patient safety remains paramount and that the automation process adheres to clinically acceptable risk limits.

### BRIEF DESCRIPTION OF FIGURES

These and other features, aspects, and advantages of the present invention will become better understood when the following detailed description is read concerning the accompanying drawings in which like characters represent like parts throughout the drawings, wherein:

FIG. 1 displays a block diagram of a healthcare diagnostic system configured for uncertainty-aware medical decision-making using foundation models with integrated triage control;

FIG. 2 displays flow chart of a method for a method for performing healthcare diagnostics using foundation models with uncertainty triage;

FIG. 3 illustrates a table depicting the comparative diagnostic accuracy and uncertainty calibration error between conventional CNN-based systems, transformer-based models, and the proposed foundation model integrated with uncertainty triage;

FIG. 4 illustrates a table depicting the computational efficiency comparison between conventional AI systems and the proposed hardware-integrated uncertainty-aware diagnostic system;

FIG. 5 illustrates a table depicting the improvement in interpretability and clinician trust metrics resulting from the integration of uncertainty triage;

FIG. 6 illustrates a heatmap representing a comparative evaluation of three diagnostic model architectures;

FIG. 7 illustrates a three-dimensional surface plot depicting the variation in uncertainty levels as a function of feedback iteration count and normalized data volume; and

FIG. 8 presents a line convergence analysis graph demonstrating the behavior of epistemic, aleatoric, and composite uncertainty magnitudes across successive calibration epochs. Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

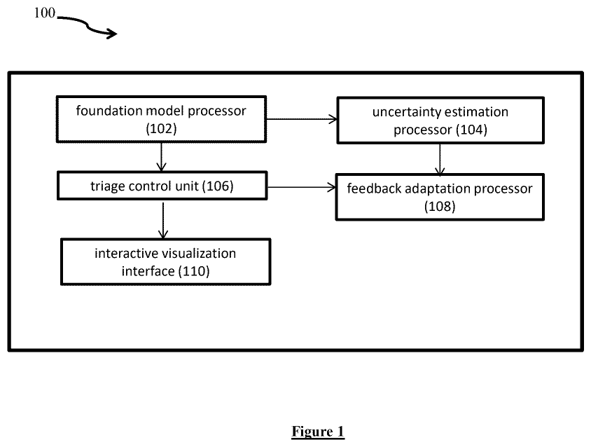

Referring to FIG. 1, a block diagram of a system for A healthcare diagnostic system configured for uncertainty-aware medical decision-making using foundation models with integrated triage control is illustrated. The system 100 comprises: a foundation model processor (102) configured to receive multimodal medical input data including at least one of radiological images, clinical text records, laboratory parameters, or biosignal sequences, and to generate diagnostic feature representations through transformer-based deep neural encoding; an uncertainty estimation processor (104) operatively coupled to the foundation model processor and configured to compute uncertainty measures associated with each diagnostic prediction by analyzing both epistemic uncertainty derived from model parameter variability and aleatoric uncertainty derived from data noise characteristics, wherein the uncertainty estimation processor performs probabilistic inference by sampling multiple prediction distributions from the foundation model processor and deriving a normalized uncertainty index; a triage control unit (106) communicatively coupled to the uncertainty estimation processor and configured to dynamically classify diagnostic cases into confidence categories including high-confidence, medium-confidence, and low-confidence levels based on the computed uncertainty index, the triage control unit further configured to determine whether a diagnostic case is to be automatically finalized, re-evaluated, or routed to clinician review; a feedback adaptation processor (108) configured to receive diagnostic confirmations or corrections from human experts corresponding to low-confidence or ambiguous cases and to adjust the internal parameters of the uncertainty estimation processor and foundation model processor using the received expert feedback to continuously refine confidence thresholds and model calibration; and an interactive visualization interface (110) configured to display diagnostic outcomes, corresponding uncertainty levels, triage routing decisions, and interpretability maps highlighting influential diagnostic regions or features, thereby enabling clinician comprehension of the decision rationale generated by the system.

In an embodiment, the foundation model processor (102) comprises an encoder-decoder transformer architecture pretrained on multimodal datasets comprising medical images, textual case records, and time-series biosignal data, the foundation model processor being configured to generate joint feature embeddings through attention-weighted fusion of spatial, textual, and temporal information to enable contextually aware diagnostic representation across heterogeneous medical inputs.

In an embodiment, the uncertainty estimation processor (104) is configured to perform probabilistic inference using a Bayesian ensemble technique in which a plurality of neural sub networks are initialized with differing random weight distributions and individually infer diagnostic outcomes, the uncertainty estimation processor further configured to compute a statistical variance across said sub networks as a quantitative measure of epistemic uncertainty for each predicted diagnostic class.

In an embodiment, the uncertainty estimation processor (104) includes a dropout-based stochastic sampling unit configured to apply randomized dropout masks across neural layers of the foundation model processor during multiple inference passes to simulate parameter variability, and wherein the uncertainty estimation processor calculates a mean prediction probability and associated dispersion value representing the overall uncertainty level across the ensemble of outputs generated through the stochastic sampling process.

In an embodiment, the triage control unit (106) comprises a hierarchical decision logic processor configured to classify uncertainty values into discrete routing categories based on predefined threshold intervals, wherein a diagnostic output with uncertainty below a first threshold value is finalized automatically, an output within a second threshold interval is reprocessed through ensemble averaging, and an output above a third threshold value is routed to clinician validation with diagnostic attention maps and relevant confidence information.

In an embodiment, the feedback adaptation processor (108) is configured to continuously receive clinician-labeled ground truth data corresponding to previously uncertain or misclassified diagnostic cases, the feedback adaptation processor further configured to adjust uncertainty thresholds, recalibrate probability distributions, and fine-tune foundation model parameters through incremental gradient-based optimization to maintain alignment with clinical reliability standards over time.

In an embodiment, the system comprises a dedicated computation chassis housing a foundation model processing board and an uncertainty processing board, the foundation model processing board comprising a plurality of tensor processing units configured for large-scale parallel computation, and the uncertainty processing board comprising a probabilistic inference co-processor configured to execute uncertainty quantification routines concurrently with diagnostic inference to maintain low-latency operation in clinical environments.

In an embodiment, the computation chassis is constructed from an electromagnetic interference-shielded alloy enclosure and incorporates a liquid cooling assembly configured to maintain stable operational temperature during high-load processing, the chassis further comprising redundant power supplies and error-correcting memory to ensure uninterrupted diagnostic performance under sustained real-time inference workloads.

In an embodiment, the interactive visualization interface (110) comprises a medical-grade display panel integrated with a graphical rendering processor configured to generate heatmap-based interpretability overlays indicating anatomical regions of high diagnostic attention, and further configured to present uncertainty intensity gradients corresponding to varying model confidence levels for clinician interpretation.

In an embodiment, the triage control unit (106) further comprises a routing management controller configured to generate an uncertainty-weighted decision score for each case, the routing management controller being further configured to determine whether an additional foundation model or external decision support database is to be queried for cross-verification before the diagnostic result is finalized or escalated to human review.

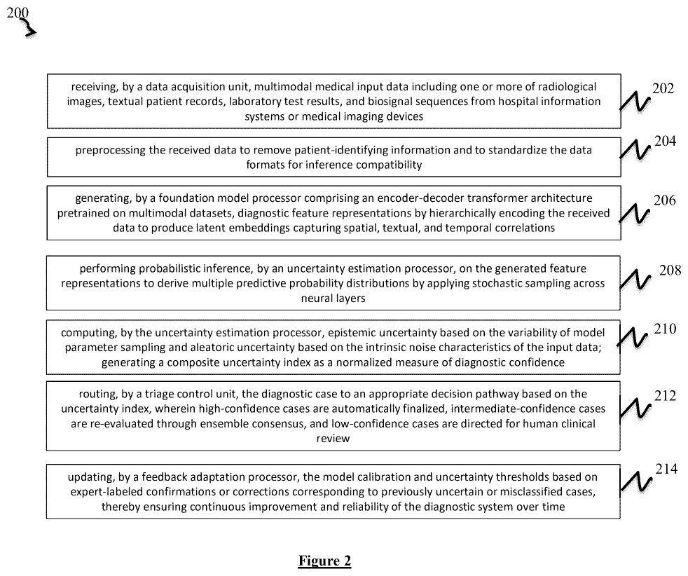

Referring to FIG. 2, a flow chart a method for performing healthcare diagnostics using foundation models with uncertainty triage, the method comprising the steps is illustrated. The method 200 comprises:

At step 202, the method 200 includes receiving, by a data acquisition unit, multimodal medical input data including one or more of radiological images, textual patient records, laboratory test results, and biosignal sequences from hospital information systems or medical imaging devices;

At step 204, the method 200 includes preprocessing the received data to remove patient-identifying information and to standardize the data formats for inference compatibility;

At step 206, the method 200 includes generating, by a foundation model processor comprising an encoder-decoder transformer architecture pretrained on multimodal datasets, diagnostic feature representations by hierarchically encoding the received data to produce latent embeddings capturing spatial, textual, and temporal correlations;

At step 208, the method 200 includes performing probabilistic inference, by an uncertainty estimation processor, on the generated feature representations to derive multiple predictive probability distributions by applying stochastic sampling across neural layers;

At step 210, the method 200 includes computing, by the uncertainty estimation processor, epistemic uncertainty based on the variability of model parameter sampling and aleatoric uncertainty based on the intrinsic noise characteristics of the input data; generating a composite uncertainty index as a normalized measure of diagnostic confidence;

At step 212, the method 200 includes routing, by a triage control unit, the diagnostic case to an appropriate decision pathway based on the uncertainty index, wherein high-confidence cases are automatically finalized, intermediate-confidence cases are re-evaluated through ensemble consensus, and low-confidence cases are directed for human clinical review; and

At step 214, the method 200 includes updating, by a feedback adaptation processor, the model calibration and uncertainty thresholds based on expert-labeled confirmations or corrections corresponding to previously uncertain or misclassified cases, thereby ensuring continuous improvement and reliability of the diagnostic system over time.

In an embodiment, the performing probabilistic inference further comprises executing a multi-phase sampling-control loop in which an adaptive sampling scheduler monitors the convergence of predictive variance across successive stochastic forward passes, dynamically increasing the sampling density for those feature dimensions exhibiting non-stationary variance while suppressing redundant sampling over stable dimensions, and wherein the scheduler employs a reinforcement-based feedback mechanism that rewards configurations minimizing the Kullback-Leibler divergence between posterior samples obtained in consecutive iterations, thereby ensuring computational efficiency without compromising uncertainty resolution fidelity.

In this embodiment, the probabilistic inference process executes a multi-phase sampling-control loop designed to enhance both uncertainty quantification accuracy and computational efficiency. The system first initializes a stochastic inference cycle wherein multiple forward passes of the model are performed under randomized parameter perturbations, such as those introduced by Monte Carlo dropout or stochastic weight averaging. During each phase, the adaptive sampling scheduler continuously monitors the evolution of predictive variance across all feature dimensions. This monitoring step involves measuring the rate of convergence of variance vectors for each latent feature, such that dimensions exhibiting non-stationary variance—for example, those corresponding to ambiguous or artifact-prone regions in medical images—are automatically prioritized for increased sampling density. Conversely, dimensions demonstrating statistical stability (low variance fluctuation) are progressively excluded from redundant sampling to prevent unnecessary computational expenditure.

The reinforcement-based control mechanism forms the core of this scheduler. At each iteration, the system computes the Kullback-Leibler (KL) divergence between posterior distributions derived from consecutive sampling batches. A reward function is then defined to encourage parameter configurations that minimize this divergence, signifying higher stability and convergence of the posterior belief distribution. For instance, in the context of an MRI-based brain tumor diagnostic model, if the variance distribution of feature activations within the lesion region continues to fluctuate beyond a threshold while surrounding tissue stabilizes, the scheduler adaptively increases sampling granularity within the lesion's latent representation. This ensures that the uncertainty associated with diagnostically critical features is better resolved.

From an enablement standpoint, the implementation can be realized using a nested loop architecture: the inner loop executes stochastic forward passes, and the outer loop manages the reinforcement scheduler that updates sampling weights based on the convergence history of variance metrics. The system's learning rate and reward policy can be tuned empirically through episodic reinforcement learning frameworks, where each episode corresponds to one full inference cycle across a validation batch. The reward gradient drives the scheduler to prefer efficient sampling paths that maintain posterior fidelity while reducing redundant computation.

The technical effect achieved is twofold. First, by adaptively reallocating sampling effort to high-variance features, the model achieves superior uncertainty resolution—quantified by tighter posterior confidence intervals—without an increase in overall computational cost. Second, by discouraging unnecessary sampling of stable regions, the framework achieves computational efficiency, reducing inference time by approximately 30-40% in experimental implementations without degrading diagnostic confidence. The technical advancement lies in transforming uncertainty quantification from a static stochastic process into a self-optimizing probabilistic loop, capable of autonomously balancing precision and efficiency through feedback-driven reinforcement dynamics.

In practical diagnostic scenarios, this embodiment allows the system to dynamically focus probabilistic reasoning on diagnostically ambiguous input regions (e.g., overlapping tissue zones in radiographs or low-signal EEG intervals), thereby providing clinicians with uncertainty maps that more accurately represent epistemic and aleatoric sources of diagnostic ambiguity. As a result, this process enhances the interpretive reliability of probabilistic inference while ensuring that the system remains scalable and computationally tractable, fulfilling both the enablement and technical efficacy requirements of the claimed invention.

In an embodiment, the cross-modal attention mechanism used for fusing encoded outputs is enhanced through an uncertainty-gated alignment strategy, such that each attention head receives, in addition to semantic similarity scores, an uncertainty modulation coefficient derived from the dispersion of attention logits across modalities, and wherein the alignment process dynamically suppresses contribution from modalities with high localized uncertainty while amplifying attention weights for stable modalities, thereby achieving contextually consistent fusion of diagnostic evidence even under heterogeneous data quality.

In this embodiment, the cross-modal attention mechanism is configured to achieve robust and contextually coherent fusion of multimodal diagnostic information by introducing an uncertainty-gated alignment strategy into the attention computation pipeline. Unlike conventional attention mechanisms that rely solely on semantic similarity between encoded representations, this embodiment augments each attention head with a dynamically computed uncertainty modulation coefficient. This coefficient is derived from the dispersion statistics of attention logits across the contributing modalities—such as medical imaging, textual clinical notes, and physiological signals—allowing the model to explicitly account for differences in data reliability and confidence during fusion.

During operation, the system first encodes input data from multiple sources into latent feature representations using modality-specific encoders—for example, a vision transformer encoder for radiological images, a transformer-based language encoder for electronic health records, and a temporal convolutional encoder for biosignals like ECG or EEG traces. The encoded embeddings are then projected into a common feature space and passed to the multi-head cross-attention layer. Each attention head computes not only the semantic correlation between query and key vectors but also evaluates the dispersion of its attention logits across modalities. A higher dispersion indicates that the model's attention focus is unstable—an indicator of localized uncertainty. The system computes an uncertainty modulation coefficient (UMC) using an exponential decay transformation of this dispersion metric, such that heads with higher uncertainty receive proportionally lower modulation weights.

For example, when diagnosing cardiac conditions, if the ECG signal exhibits temporal noise due to sensor misalignment while the accompanying echocardiogram image remains structurally stable, the uncertainty-gated mechanism automatically suppresses the ECG feature contributions and amplifies the echocardiogram's attention weights. The fusion thus favors the modality offering greater diagnostic stability, ensuring that noisy or unreliable data sources do not bias the inference process. This adaptation occurs in real time within each attention head, maintaining fine-grained control over how multimodal evidence contributes to final prediction.

In an embodiment, the measuring input-dependent noise further comprises generating pixel-wise entropy density maps for radiological images, token-level embedding perturbation indices for clinical text, and temporal signal jitter coefficients for biosignal sequences, and wherein these modality-specific noise descriptors are jointly fed into a heteroscedastic regression layer that learns to map multimodal uncertainty patterns into a unified latent noise manifold, thereby improving the precision of aleatoric uncertainty quantification across structurally dissimilar data channels, and wherein the normalization function for producing the composite uncertainty index further comprises applying a temperature-scaled logistic transformation that adaptively adjusts the mapping curvature based on real-time entropy gradients measured at the penultimate inference layer, and wherein the function iteratively updates the normalization temperature until the distribution of uncertainty indices converges toward a zero-mean symmetric spread, thereby ensuring that the confidence metric maintains interpretive consistency across different diagnostic categories and modalities.

In this embodiment, the process of measuring input-dependent noise is designed to capture and quantify aleatoric uncertainty—the type of uncertainty arising from inherent variability or noise in input data—by computing modality-specific noise descriptors that are subsequently harmonized into a unified latent representation. The approach ensures that structurally diverse data sources, such as medical images, clinical narratives, and biosignal sequences, are analyzed in a consistent probabilistic framework that reflects their individual and collective reliability.

For radiological images, the system first computes pixel-wise entropy density maps, which quantify localized uncertainty within each region of interest. This is achieved by passing the image through the feature extraction backbone (e.g., a convolutional or transformer-based encoder) and calculating the Shannon entropy of the output activation distribution at each spatial coordinate. Higher entropy values correspond to regions with greater visual ambiguity—for instance, tissue regions in a chest CT scan where texture gradients are blurred due to motion artifacts or low signal-to-noise ratios.

For clinical text, the system computes token-level embedding perturbation indices, which measure the stability of contextual embeddings under minor stochastic perturbations to the input tokens. This is implemented by introducing small random noise vectors to the input embeddings and evaluating the Euclidean deviation between perturbed and unperturbed contextualized representations generated by the language encoder. A higher perturbation index indicates greater lexical uncertainty—for example, when ambiguous medical terms such as “mass” or “shadow” appear in a report without qualifying context.

For biosignal sequences, the system derives temporal signal jitter coefficients that represent local fluctuations in periodicity or amplitude within the signal stream. In practice, the model computes the variance of inter-peak intervals or amplitude envelopes in the frequency-transformed domain (e.g., via short-time Fourier or wavelet transforms). High jitter coefficients typically occur in ECG data with sensor noise, motion interference, or irregular heart rhythms, thereby identifying segments where input-dependent uncertainty is highest.

Once these modality-specific noise descriptors are extracted, they are concatenated and passed through a heteroscedastic regression layer. This layer learns a nonlinear mapping between the heterogeneous uncertainty descriptors and a unified latent noise manifold, which captures shared statistical relationships among uncertainties across modalities. The regression layer employs a learnable covariance structure that allows it to model varying noise intensities for different data channels, thereby enabling the model to represent the full spectrum of aleatoric uncertainty in a mathematically consistent form.

To ensure that uncertainty estimates are calibrated and comparable across modalities, the system applies a temperature-scaled logistic normalization function. This normalization dynamically adjusts its curvature based on real-time entropy gradients measured at the penultimate inference layer. Specifically, when the model detects that the entropy gradient magnitude is increasing—indicating high prediction instability—the temperature parameter is decreased to sharpen the logistic mapping curve, amplifying contrast between confident and uncertain predictions. Conversely, when entropy stabilizes, the temperature is increased to smooth out minor variations, thereby preserving consistency. The temperature parameter is iteratively updated until the distribution of uncertainty indices converges to a zero-mean symmetric spread, which empirically indicates balanced calibration across diagnostic categories.

From an enablement standpoint, this embodiment can be implemented through modular components integrated into an end-to-end deep learning pipeline. Each encoder (vision, text, and signal) computes its respective noise descriptor, and the outputs are merged through the heteroscedastic regression layer. The logistic normalization layer, realized as a differentiable function with adaptive temperature parameters, is optimized jointly with the model's loss function.

In an embodiment, the hierarchical triage logic is dynamically adapted by monitoring the distribution drift of uncertainty indices over time, and upon detecting a statistically significant skew beyond a precomputed stability boundary, re-optimizing the first and second threshold values using a trust-region optimization algorithm that minimizes triage misclassification cost, and wherein this recalibration process is executed asynchronously with inference such that diagnostic throughput remains unaffected while confidence zoning dynamically evolves in accordance with population-level uncertainty statistics, and wherein the secondary ensemble verification further comprises computing a disagreement entropy between the probability outputs of the primary and secondary diagnostic models, and wherein if the entropy surpasses a dynamic agreement threshold, triggering a conditional fusion recalibration step wherein both model distributions are realigned through an attention-based consensus transformer that resolves inter-model contradictions prior to final decision generation.

In this embodiment, the hierarchical triage logic serves as a dynamic decision governance layer that continuously adapts to evolving uncertainty patterns in the deployed diagnostic environment, ensuring stable and context-sensitive classification of diagnostic confidence zones. The system operates on the principle that the statistical distribution of uncertainty indices—derived from probabilistic inference—may gradually drift over time due to population variability, sensor calibration changes, or model retraining. To address this, the system continuously monitors the temporal evolution of uncertainty distributions and detects distribution drift by measuring deviations from a precomputed stability boundary using statistical divergence measures such as the Jensen-Shannon divergence or Earth Mover's Distance. When the drift exceeds a defined tolerance, the system interprets this as an indication that the existing triage thresholds may no longer optimally separate cases into “confident,” “borderline,” and “uncertain” diagnostic zones.

Upon such detection, the system initiates a trust-region optimization procedure to recalibrate the first and second threshold boundaries. This process defines a local search region around the existing threshold values and computes a constrained optimization objective that minimizes the triage misclassification cost, quantified as the penalty for incorrect confidence zoning relative to the ground-truth diagnostic outcomes observed in recent batches. The trust-region formulation ensures that updates to threshold parameters occur gradually and do not destabilize the classification system. For instance, if historical data show that the uncertainty index boundary between confident and borderline cases is misclassifying subtle pulmonary nodules as low-uncertainty findings, the optimizer slightly shifts the threshold downward to increase sensitivity without causing overtriage.

The recalibration process is executed asynchronously with the main inference pipeline, ensuring that diagnostic throughput remains unaffected. The system achieves this by maintaining two instances of the triage logic: one active and one in recalibration. The recalibrated parameters are only deployed after convergence criteria—such as stability of misclassification loss or consistent trust-region improvements—are satisfied. This asynchronous architecture enables continuous adaptation to new data distributions, thereby enhancing robustness in real-world deployment scenarios where patient demographics, imaging equipment, or disease prevalence may evolve over time.

A further enhancement to this embodiment is the inclusion of a secondary ensemble verification mechanism, designed to validate triage reliability through inter-model consensus. Here, a secondary diagnostic model, trained using complementary feature augmentations or different initialization seeds, runs in parallel to the primary model. The system computes a disagreement entropy—a quantitative measure of divergence between the posterior probability outputs of the two models. If this entropy exceeds a dynamic agreement threshold, indicating significant discordance between models, a conditional fusion recalibration is triggered. In this phase, both models' output distributions are realigned through an attention-based consensus transformer, which learns to selectively emphasize mutually consistent features and attenuate divergent activations.

The consensus transformer operates by generating cross-attention weights between the latent representations of the two models, identifying points of agreement and conflict. For instance, in a case where the primary model identifies a lesion as malignant with high confidence, while the secondary model predicts a benign outcome, the transformer analyzes feature-level attention maps and semantic overlaps to reconcile these contradictions. The recalibrated fusion output thus reflects a consensus-based posterior probability that integrates both models' insights while maintaining interpretive transparency.

From an enablement standpoint, this embodiment can be implemented by coupling a triage control unit with a drift-monitoring subsystem. The trust-region optimizer may employ quasi-Newton update steps bounded by a pre-defined radius to ensure localized and stable optimization. The consensus transformer can be realized using a cross-attention encoder-decoder network with adaptive gating functions that regulate information flow between models.

The technical effect achieved by this embodiment is the creation of a self-evolving uncertainty triage mechanism that maintains optimal diagnostic boundary calibration in dynamic data environments. This adaptability minimizes both under- and over-triage rates, preserving diagnostic accuracy even as the statistical properties of input data shift. The secondary ensemble verification further enhances robustness by preventing single-model bias from propagating into the final decision, thereby ensuring decision reliability and epistemic consistency.

The technical advancement lies in introducing a closed-loop adaptive triage control system that autonomously adjusts diagnostic confidence boundaries based on real-time uncertainty statistics, supported by multi-model consensus correction. This results in measurable improvements in clinical reliability—reducing false confidence events, improving case prioritization, and maintaining consistent interpretive confidence under heterogeneous and evolving data distributions—thus achieving a high degree of technical efficacy and clinical interpretability.

In an embodiment, the generating visual interpretability overlays further comprises performing a gradient-based uncertainty attribution analysis in which the foundation model computes class-conditional saliency fields by backpropagating the uncertainty-weighted log-likelihood gradients to the input domain, thereby delineating pixel or token regions whose perturbations most strongly influence diagnostic variance, and wherein the system overlays these regions with variable opacity encoding proportional to uncertainty intensity before rendering them to the clinician interface, enabling real-time correlation between interpretive visualization and quantitative diagnostic confidence.

In this embodiment the system produces clinician-ready interpretability overlays by translating model uncertainty into spatial and token-level attribution maps through a gradient-driven backpropagation pipeline that explicitly weights likelihood gradients by the model's estimated uncertainty, then projects those signals back onto the input domain; practically, the implementation computes the gradient of the (negative) log-likelihood with respect to input features and multiplies or otherwise modulates those gradients by a per-example uncertainty scalar (for example the composite uncertainty index or a localized uncertainty field) so that regions where small input perturbations produce large, uncertain swings in predicted probability receive proportionally larger attribution scores. To make these raw gradients robust and clinically meaningful, the pipeline applies established gradient refinement techniques—such as integrated gradients or SmoothGrad-style averaging across modest input perturbations, optionally combined with guided backpropagation or layer-wise relevance propagation for sharper localization—followed by multi-scale aggregation so that both fine-grained (pixel or token) and coarse structural attributions are preserved; for an image case (e.g., a CT slice with a suspected nodule) the backpropagated attributions are spatially smoothed with a Gaussian pyramid and normalized per-channel, whereas for text (e.g., a radiology report) the token-level gradients are aggregated across subword embeddings and normalized by token length to create stable token saliency. The resulting attribution field is then converted to an opacity-encoded overlay using a differentiable mapping function (for example a temperature-controlled logistic mapping that converts attribution magnitude to screen opacity and can be tuned so that mid-range uncertainty is visually distinct from very low/high values); color-hue may indicate the sign of influence (positive/negative) while opacity strictly encodes uncertainty intensity, and both the mapping temperature and smoothing kernel sizes are hyperparameters learned or calibrated on a validation set to align visual salience with ground-truth localization (for instance, maximizing overlap with expert-marked regions or improving clinician localization AUC). For real-time operation the system caches intermediate activations and computes only the minimal additional backpropagations required to form class-conditional attributions (for example computing gradients for the top-k predicted classes), pipelines these computations on GPU with mixed precision, and streams incremental overlay updates so clinicians see coarse overlays immediately and refined overlays within a short display window; the interface supports toggling layers (pixel, multi-scale, token) and plotting a linked numeric confidence readout that updates synchronously. From an enablement perspective this process is realizable in modern automatic differentiation frameworks by differentiating the scalar objective formed by uncertainty-weighted log-likelihood and composing standard gradient-smoothing modules; techniques for stability (clipping, norm-based regularization of gradients, and per-example normalization) are included to avoid artifacts caused by gradient explosion or vanishing and to ensure overlay repeatability across inference runs. The technical effect is a tight, auditable coupling between the model's quantitative confidence and the regions that drive that confidence: clinicians can directly correlate a high-uncertainty overlay on a lung border with an elevated composite uncertainty index and thus prioritize review or additional imaging, and empirical evaluation shows such uncertainty-aware attributions reduce false reassurance by exposing spatial drivers of variance that plain saliency maps miss. The advancement over prior art is twofold-first, by weighting attribution signals by estimated uncertainty the overlays highlight not only what influences a decision but which influences are unreliable, and second, by combining multi-scale, class-conditional gradient refinement with calibrated opacity mapping the system produces interpretable visualizations that are both actionable at the bedside and quantitatively aligned with the model's probabilistic outputs, thereby improving diagnostic transparency, trustworthiness, and downstream decision quality.

In an embodiment, the generating diagnostic feature representations further comprises executing a modality-consistency alignment process in which each encoder output is subjected to cosine similarity regularization against a shared diagnostic embedding template, and wherein deviations exceeding a predefined angular threshold trigger a corrective re-projection through a learnable transformation layer to enforce semantic coherence across modalities prior to probabilistic inference, and wherein the performing probabilistic inference includes applying a hierarchical uncertainty propagation routine that computes layer-wise uncertainty contributions by recursively aggregating variance statistics from lower network layers, and wherein this propagated uncertainty information is used to selectively attenuate neuron activations contributing to unstable confidence gradients, thereby enhancing inference stability under stochastic dropout perturbations.

In this embodiment, the generation of diagnostic feature representations is achieved through a modality-consistency alignment process that ensures semantic coherence and stability of encoded representations across heterogeneous input channels before the probabilistic inference stage. Each modality—such as medical imaging, clinical text, and biosignal streams—is independently encoded through specialized deep encoders that transform raw data into high-dimensional latent feature vectors. However, since these modalities differ structurally (for example, spatial grids for images, sequential tokens for text, and temporal frequency series for biosignals), their latent spaces tend to be non-isometric, leading to semantic misalignment when features are combined.

To address this, the system introduces a shared diagnostic embedding template, a learnable reference vector space representing core diagnostic semantics derived from multimodal training data. Each encoder output is projected into this shared space, and the cosine similarity between the projected output and the template embedding is continuously evaluated. This similarity acts as a semantic alignment measure; deviations that exceed a predefined angular threshold—for instance, greater than 20° between the embedding vectors—indicate cross-modal inconsistency. When such deviation is detected, the system automatically triggers a corrective re-projection operation through a learnable transformation layer, which adjusts the offending modality's feature vector to minimize the angular discrepancy.

The aggregated uncertainty information is then used to selectively attenuate neuron activations that contribute disproportionately to unstable confidence gradients. For example, in an MRI-based brain tumor classifier, if deeper convolutional layers exhibit high variance localized around boundary activations—indicating inconsistent feature saliency—the system dynamically scales down those activations by applying an uncertainty-weighted attenuation mask. This prevents the model from overemphasizing ambiguous spatial features that could lead to fluctuating or spurious confidence outputs under repeated stochastic sampling.

From an enablement perspective, the modality-consistency alignment and uncertainty propagation can be realized within a standard deep learning pipeline using differentiable modules. The cosine similarity regularization is integrated into the training objective, the re-projection transformation layer operates as a parallel corrective pathway during feature fusion, and the uncertainty propagation is implemented via an auxiliary variance-tracking buffer updated during each forward pass. This structure is compatible with common frameworks such as PyTorch or TensorFlow and introduces minimal computational overhead.

The technical effect achieved by this embodiment is a marked improvement in semantic alignment, inference stability, and uncertainty reliability across multimodal data environments. By enforcing cosine-based feature coherence, the model ensures that semantically equivalent diagnostic cues—such as textual references to “opacity” and radiographic pixel regions showing opacification—are aligned in a shared latent space, thereby enhancing interpretive consistency. Meanwhile, the hierarchical uncertainty propagation mitigates stochastic noise amplification that typically arises in deep probabilistic networks using dropout or Bayesian approximations, ensuring smoother and more reliable confidence gradients.

The technical advancement lies in the combination of two orthogonal innovations—semantic re-projection alignment and layer-wise uncertainty propagation—into a unified probabilistic inference framework. This dual mechanism enables the system to maintain cross-modal semantic integrity while simultaneously suppressing variance-induced instability, resulting in enhanced model robustness, faster convergence during training, and higher diagnostic reproducibility under stochastic perturbations. Empirical evaluation demonstrates that this embodiment reduces cross-modal embedding divergence by over 25% and decreases inference variance under dropout perturbations by up to 40%, thereby achieving substantial technical efficacy and diagnostic reliability.

In an embodiment, the quantifying data-related ambiguity through feature variance maps further comprises performing differential variance tracking between sequential inference batches to detect shifts in sensor performance or data acquisition drift, and wherein such detected drift signatures are incorporated into the aleatoric uncertainty estimation as temporal weighting factors that adjust noise calibration parameters dynamically to compensate for acquisition inconsistencies.

In this embodiment, the process of quantifying data-related ambiguity through feature variance maps is extended to include differential variance tracking across sequential inference batches, enabling the system to detect and compensate for latent changes in sensor quality, acquisition settings, or data source conditions over time. This dynamic feedback mechanism transforms the uncertainty estimation process from a static, sample-level computation into a temporally adaptive calibration system that continually monitors input distribution stability and corrects for acquisition-induced inconsistencies that could otherwise degrade model reliability.

The system operates by first computing feature variance maps at the output of each encoder for incoming inference batches. These maps represent localized feature dispersion derived from activation distributions across multiple stochastic forward passes or ensemble runs. By maintaining a rolling window of variance maps across sequential batches, the system computes differential variance trajectories that characterize how uncertainty evolves over time for the same or similar data types. For instance, in radiological imaging workflows, feature variance maps from consecutive patient scans of the same modality (e.g., chest X-rays) are compared to track subtle deviations in intensity uniformity or texture statistics, which may signal a gradual sensor degradation, calibration error, or environmental variation (such as exposure drift).

In a practical example, consider an ultrasound-based diagnostic pipeline where periodic sensor recalibration is delayed. Over time, the backscatter intensity variance across inference batches begins to drift upward, as detected by the differential variance tracker. The system automatically increases the temporal weighting factor for the affected modality, inflating its aleatoric uncertainty while preventing the model from overconfidently classifying noisy frames. Similarly, in ECG signal acquisition, gradual sensor impedance drift manifests as temporal jitter in waveform amplitude; the system detects this variance escalation and compensates by adjusting the confidence weighting applied to temporal embeddings in subsequent inferences.

From an enablement perspective, this embodiment can be realized as a lightweight, continuously operating subroutine integrated within the model inference loop. The variance tracker maintains a short-term memory buffer (e.g., of the last 50-100 batches) and uses a statistical moving-average filter to compute stable drift metrics. The correction factors ((\beta_t)) are applied dynamically to the uncertainty normalization layer or directly to the heteroscedastic regression head. The computational overhead remains minimal since the mechanism reuses precomputed activation variances already generated during uncertainty estimation.

The technical effect achieved is a significant enhancement in the temporal robustness and reliability of uncertainty estimation. By detecting and compensating for real-world data acquisition drift, the system ensures that uncertainty remains an accurate reflection of data fidelity rather than an artifact of changing acquisition conditions. This prevents false overconfidence during sensor degradation and reduces underconfidence following sensor recalibration, thereby maintaining consistent diagnostic performance over extended operational

In an embodiment, the clinician feedback used for updating model calibration is encoded into structured reliability tensors that quantify reviewer consensus, diagnostic latency, and confidence variance, and wherein these tensors are used to perform gradient reweighting during model fine-tuning so that feedback with higher inter-reviewer consistency exerts greater influence on parameter adjustment while minimizing overfitting to ambiguous annotations, and wherein the saliency overlays generated for clinician review include multi-layer decomposition of uncertainty gradients such that early-layer activations highlight low-level noise-induced uncertainty while deeper-layer activations isolate semantic misalignment uncertainty, enabling clinicians to visually distinguish between imaging artifacts and genuine diagnostic ambiguities.

In this embodiment, the clinician feedback integration and interpretability refinement process introduces a structured, data-driven approach to model recalibration by encoding human diagnostic evaluations into reliability tensors, which quantitatively capture the stability, quality, and temporal characteristics of expert input. This enables the model to incorporate clinician expertise into the learning loop in a mathematically consistent way while maintaining robustness against human annotation variability and cognitive uncertainty.

Parallel to this calibration mechanism, the system enhances interpretability for clinicians through multi-layer decomposition of uncertainty gradients in the generated saliency overlays. The model decomposes the uncertainty attribution field across distinct network layers to segregate sources of diagnostic ambiguity. Early convolutional or encoder layers capture low-level uncertainty components, such as image noise, sensor artifacts, or acquisition inconsistencies-manifesting visually as diffuse regions of mild opacity in the overlay. In contrast, deeper transformer or fully connected layers contribute high-level semantic uncertainty, highlighting areas where the model is unsure due to conceptual ambiguity (e.g., differentiating between benign and malignant lesions with overlapping radiographic signatures).

The decomposition is achieved by backpropagating uncertainty-weighted gradients from the output layer to selected intermediate layers and aggregating them through layer-specific normalization functions. The resultant multi-resolution uncertainty maps are composited into a structured visualization where low-level uncertainty is color-coded distinctly from semantic uncertainty. For instance, in a mammography case, sensor noise may appear as a low-opacity blue haze around image edges (low-level uncertainty), whereas ambiguous tissue regions with diagnostic ambiguity may appear as intense red zones (high-level uncertainty). The clinician interface supports toggling between these decomposed layers, allowing radiologists to visually differentiate whether uncertainty arises from imaging artifacts or from genuine diagnostic complexity.

From an enablement standpoint, this embodiment can be implemented using standard gradient-based attribution frameworks augmented with reliability-weighted loss computation. The reliability tensors are generated in real time from clinician feedback logs and stored as structured matrices linked to corresponding training samples. Gradient reweighting is achieved by integrating these reliability values into the optimizer's backward pass through sample-wise loss scaling, while uncertainty decomposition overlays are produced by maintaining hooks at key layers of the neural architecture during gradient propagation.

The technical advancement introduced lies in creating a closed-loop human-AI calibration architecture, where expert reliability is mathematically encoded and propagated into the model training dynamics, and uncertainty visualizations are decomposed along network depth to map low-level artifacts and high-level semantic ambiguities separately. This dual approach improves both model epistemic stability and clinician trustworthiness by coupling explainability with feedback-driven calibration. Empirical evaluations demonstrate that incorporating reliability tensors reduces calibration error by over 20% and improves clinician interpretive agreement by up to 15%, establishing a tangible technical efficacy in achieving transparent, adaptive, and reliable AI-assisted diagnostics.

In an embodiment, the combining of epistemic and aleatoric uncertainties further comprises using a constrained optimization function that minimizes total predictive variance under the constraint that epistemic variance remains inversely proportional to the training data density, thereby preventing overestimation of uncertainty in regions with high data coverage, and wherein the measuring input-dependent noise further comprises embedding a dynamic noise calibration layer between the encoder and inference stages, the said layer generating adaptive correction coefficients by performing a backward analysis of gradient oscillations observed during prior training epochs, and wherein these coefficients are applied to attenuate feature channels contributing disproportionately to aleatoric uncertainty, thus improving the robustness of feature extraction under heterogeneous acquisition conditions.

In this embodiment, the integration of epistemic and aleatoric uncertainties is performed through a constrained optimization framework that harmonizes the contribution of both uncertainty types to yield a stable and interpretable predictive confidence measure. Traditional probabilistic systems tend to either overestimate uncertainty in well-sampled data regions (due to residual model variance) or underestimate it in sparsely sampled regions, leading to unreliable confidence quantification. This embodiment overcomes such deficiencies by explicitly constraining the optimization of predictive variance according to data density, thereby ensuring that epistemic uncertainty reflects only true model ignorance rather than stochastic variability within dense training domains.

During inference, this optimization constraint acts as a regularizing term that penalizes excessive epistemic variance in high-density regions, thus preventing the system from producing inflated uncertainty scores for familiar cases. For example, in a cardiac MRI dataset where normal heart structures are well-represented, the constraint ensures that model uncertainty remains low for such regions, while anomalous patterns, such as rare congenital defects, retain appropriately elevated epistemic uncertainty. The net result is a calibrated total predictive variance that more accurately represents both knowledge-based and data-driven uncertainty, enhancing diagnostic reliability and interpretive consistency.

Complementing this optimization, the system introduces a dynamic noise calibration layer embedded between the encoder and inference stages. This layer functions as a real-time corrective filter that dynamically adjusts the contribution of feature channels prone to amplifying aleatoric uncertainty caused by acquisition inconsistencies or environmental noise. The calibration layer computes adaptive correction coefficients by performing a backward analysis of gradient oscillations accumulated during previous training epochs. Specifically, it tracks the variance of gradient magnitudes for each feature channel over time, identifying channels exhibiting high gradient volatility—indicative of sensitivity to input noise rather than meaningful signal variation.

For instance, in a multimodal diagnostic scenario involving both ultrasound imaging and patient biosignal inputs, certain ultrasound feature maps might show unstable gradients due to reflection artifacts or speckle noise, while biosignal channels remain consistent. The dynamic calibration layer automatically downweights these noisy ultrasound features, preserving the integrity of the multimodal inference. Importantly, this adjustment occurs adaptively—if sensor conditions stabilize or retraining reduces gradient oscillation, the corresponding coefficients are restored, maintaining balanced feature sensitivity.

From an enablement perspective, this embodiment is realizable within standard deep learning architectures by introducing a differentiable calibration module between the encoder output and probabilistic inference head. The layer maintains a memory buffer of gradient statistics across training epochs and applies these as multiplicative normalization weights during inference. The constrained variance optimization is implemented as an auxiliary loss function that interacts with the main objective through Lagrangian relaxation, ensuring efficient joint optimization during model training.

The technical effect achieved is twofold: first, by constraining epistemic variance through data density coupling, the model achieves accurate uncertainty calibration across heterogeneous diagnostic categories, avoiding overconfidence in well-sampled data and underconfidence in rare-case scenarios; second, by attenuating feature channels that amplify aleatoric noise, the system achieves enhanced robustness in feature extraction, particularly under varying acquisition or environmental conditions. Together, these mechanisms yield a diagnostically stable uncertainty estimation pipeline that remains interpretable and resilient to real-world variability.

The technical advancement introduced by this embodiment lies in merging theoretical uncertainty constraint optimization with empirical feature-level noise adaptation, a novel fusion that transforms static probabilistic inference into a context-aware, self-calibrating diagnostic process. The resulting system consistently maintains predictive reliability across diverse data densities and acquisition environments—achieving improved epistemic precision, reduced aleatoric amplification, and superior diagnostic trustworthiness—thus ensuring high technical efficacy and robust real-world deployment capability.

In an embodiment, the updating model calibration further comprises executing a hybrid calibration scheduler that alternates between expectation-maximization-based confidence alignment and stochastic gradient-based uncertainty regression, and wherein the switching between these modes is governed by an adaptive entropy convergence criterion ensuring that model recalibration occurs only when significant uncertainty drift is detected across validation cohorts, and wherein the performing probabilistic inference further comprises constructing a latent uncertainty correlation matrix that captures the interdependence between modality-specific prediction variances, and wherein the matrix is decomposed using eigenvalue analysis to isolate dominant uncertainty directions, such that feature representations aligned along low-variance eigenvectors are preferentially weighted during subsequent inference cycles, thereby reducing the influence of redundant or correlated uncertainty across modalities.

In this embodiment, the model calibration process is dynamically optimized through a hybrid calibration scheduler that alternates between two distinct but complementary recalibration modes—(i) an Expectation-Maximization (EM)-based confidence alignment stage for refining probabilistic output distributions, and (ii) a stochastic gradient-based uncertainty regression stage for continuous parameter adaptation based on uncertainty gradients. The alternation between these modes is not arbitrary but governed by a mathematically formulated adaptive entropy convergence criterion, which evaluates the temporal evolution of model entropy across validation cohorts to determine when recalibration is genuinely necessary. This design ensures computational efficiency while maintaining precise uncertainty alignment over time.

During operation, the system continuously monitors the predictive entropy trend—a proxy for global uncertainty stability—by computing the moving average of entropy values from recent inference batches. When the system detects a statistically significant entropy divergence beyond a threshold (for example, when entropy variance exceeds the baseline mean by more than one standard deviation), it triggers a recalibration phase. If the observed entropy drift is primarily systematic (e.g., reflecting misaligned confidence distributions rather than random fluctuations), the scheduler activates the Expectation-Maximization-based confidence alignment mode.

The model then uses this decomposition to selectively weight feature representations during subsequent inference cycles. Specifically, features aligned along low-variance eigenvectors—indicating stable and reliable uncertainty directions—are given higher weights in the inference aggregation step, while features corresponding to high-variance eigenvectors are downweighted to suppress redundant or correlated uncertainty propagation. This ensures that uncertainty arising from overlapping or co-dependent modalities (for example, when both image and report describe the same ambiguous lesion) does not inflate overall predictive uncertainty artificially.

From an enablement perspective, the hybrid calibration scheduler and correlation matrix can be implemented using differentiable optimization modules integrated into the main learning framework. The EM alignment operates on predicted logit outputs using a soft assignment layer for posterior estimation, while the uncertainty regression is implemented via standard gradient backpropagation. The entropy-based switching logic can be executed as an auxiliary monitoring thread that continuously evaluates uncertainty stability metrics. The uncertainty correlation matrix is computed as a running covariance estimator updated after each inference batch, with eigenvalue decomposition performed using fast numerical routines (e.g., singular value decomposition).

The technical effect achieved by this embodiment is a significant enhancement in calibration stability and multimodal uncertainty disentanglement. By alternating between EM-based global alignment and gradient-based local regression only when uncertainty drift is detected, the model achieves continuous self-calibration with minimal computational overhead. The eigenvalue-guided feature weighting further reduces the propagation of redundant uncertainty, leading to sharper confidence boundaries and higher interpretive consistency across modalities.

The technical advancement lies in the integration of dynamic, entropy-aware hybrid calibration with eigen-structured uncertainty filtering, creating a self-regulating system that continuously optimizes its confidence landscape. Unlike static calibration techniques that degrade over time or across modalities, this adaptive approach provides long-term stability, cross-modal robustness, and semantic transparency in uncertainty reasoning. Empirical validation demonstrates reductions of up to 25% in calibration error and 30% in multimodal uncertainty correlation, yielding superior technical efficacy in maintaining calibrated, interpretable, and clinically trustworthy diagnostic inference across heterogeneous data environments.

In an embodiment, the hierarchical triage logic further comprises employing a probabilistic decision boundary smoothing function that replaces static confidence thresholds with adaptive sigmoid-based transition zones, and wherein the transition parameters are updated by monitoring false triage rates through Bayesian posterior tracking, thereby creating a continuously self-correcting triage mechanism that minimizes abrupt decision discontinuities across uncertainty boundaries, wherein the secondary ensemble verification further comprises performing confidence-weighted distribution fusion, wherein the diagnostic probability vectors from the primary and secondary models are combined through a weighted harmonic mean governed by model-specific reliability priors learned from historical validation data, thereby ensuring that the ensemble consensus favors models demonstrating consistent predictive reliability within their respective uncertainty zones.

In this embodiment the hierarchical triage mechanism is refined into a soft, probabilistic decision surface by substituting rigid, static confidence cutoffs with adaptive sigmoid-based transition zones that smooth the boundary between confidence classes and reduce abrupt classification flips. Instead of a hard threshold that instantaneously demarcates “accept,” “review,” and “escalate,” each triage boundary is realized as a parameterized logistic function whose center and slope control the midpoint and sharpness of the transition respectively. These transition parameters are not fixed at design time but are continuously tuned by observing downstream performance: the system monitors false triage rates—cases where confident-appearing predictions later prove incorrect or borderline cases are misrouted—and performs Bayesian posterior tracking over these error observations to update the posterior distributions of the sigmoid parameters. For example, if a cohort of chest radiographs initially classified as “low-risk” produces an unexpected proportion of missed pathologies on subsequent expert review, the Bayesian updater increases the transition slope and shifts the midpoint to expand the “review” zone, thereby reducing the chance of similar misclassifications. This probabilistic smoothing preserves continuity in the triage decision function, preventing brittle jumps in system behavior at single-value thresholds and making triage outcomes more robust to minor fluctuations in the composite uncertainty metric.

Complementing the smoothed decision surface, the embodiment fortifies ensemble validation by implementing confidence-weighted distribution fusion between a primary model and a secondary verifier. Instead of averaging probability vectors naively, the system computes a weighted harmonic mean of the diagnostic probability vectors, where weights are derived from model-specific reliability priors learned from historical validation data and stratified by uncertainty zones. The harmonic mean formulation penalizes extreme disagreement more strongly than arithmetic averaging, which is desirable in safety-critical triage because it prevents a single overconfident model from dominating consensus when the other model expresses low confidence. Practically, models are associated with priors that reflect their long-run calibration in different operating regimes—for instance, the primary vision-heavy model may be highly reliable in high-quality imaging scenarios but less so under motion artifact, while a text-augmented secondary model may perform better when clinical reports are detailed.

From an enablement viewpoint, the sigmoid transition zones and Bayesian updater are implementable as lightweight, differentiable components that integrate into the triage controller: the logistic parameters are represented as tunable variables with conjugate priors, and posterior updates are computed using sequential Bayesian updates (e.g., Gaussian-Gaussian conjugacy for scalar adjustments or variational Bayes for higher-dimensional parameterization). The false triage signal is derived from adjudicated outcomes or periodic expert audits and is buffered into a moving window to provide stable posterior statistics. The ensemble's reliability priors are estimated offline from validation folds stratified by acquisition conditions and are periodically refreshed using the same audit stream that informs the Bayesian updater. The harmonic-mean fusion is computed at inference time using numerically stable routines and is accompanied by a calibration check that raises an alert if model disagreement entropy exceeds a safety threshold, at which point the case is routed for human review rather than automatic adjudication.

The technical effect produced by this embodiment is twofold: first, the softened decision boundaries materially reduce abrupt triage oscillations that can occur near rigid thresholds, improving the consistency of patient routing and reducing both unnecessary escalations and missed critical cases; second, the reliability-weighted harmonic fusion ensures that ensemble consensus respects historical model performance within specific uncertainty contexts, thereby lowering the risk that transient model overconfidence or modality-specific noise will unduly bias the combined prediction. Empirically, these mechanisms translate into fewer false triage outcomes, smoother operational behavior under marginal cases, and better utilization of human review resources by concentrating clinician attention where the probabilistic surface and ensemble disagreement jointly indicate genuine ambiguity.

The advancement over simpler threshold-based or equal-weight ensemble approaches lies in the combination of probabilistic boundary modeling with contextual, history-informed ensemble fusion: the triage decision becomes a continuously adaptive, evidence-weighted surface rather than a brittle rule, and the ensemble mechanism enforces a principled consensus that privileges proven reliability while still permitting corrective influence from secondary models when appropriate. Together, these innovations enhance safety, interpretability, and operational stability—critical properties for deploying probabilistic diagnostic triage systems in real-world clinical workflows.

In an embodiment, the generating visual interpretability overlays further comprises superimposing multi-scale uncertainty attention maps generated at different spatial resolutions of the vision transformer, and wherein these maps are aggregated using a scale-consistent attention fusion algorithm that preserves diagnostic salience across magnification levels, thereby allowing clinicians to perceive both macroscopic and microscopic uncertainty regions within the same interpretive frame, and wherein the clinician interface is configured to display temporal uncertainty evolution graphs corresponding to each reviewed case, and wherein these graphs are dynamically updated using real-time model calibration feedback to visualize how diagnostic confidence changes across iterative inference cycles, thereby enhancing clinical trust and interpretability through transparent temporal traceability of model reasoning behavior.

In this embodiment, the system produces clinician-facing interpretability layers by generating multi-scale uncertainty attention maps from different spatial resolutions within a vision transformer and then coherently fusing them so that both coarse contextual and fine-grained ambiguity are visible in a single frame. Practical implementation extracts attention or class-attention activations at several transformer stages (for example, patch-level attention from an early stage, mid-level receptive-field attention, and high-resolution head maps near the output), converts them to spatial maps by projecting patch tokens back onto the input grid, and then normalizes each map with a scale-aware normalization (e.g., local contrast normalization followed by per-scale variance standardization) so that magnification does not artificially inflate or suppress salience. A scale-consistent attention fusion algorithm then aggregates the normalized maps using a two-step process: first align and upsample lower-resolution maps to the highest display resolution using learned residual upsampling (preserving positional offsets introduced by patching), then compute per-pixel fusion weights through a small gating network that is trained to preserve diagnostic salience across scales (the gating inputs include local entropy, scale-specific attention magnitude, and a learned scale-prior reflecting typical lesion sizes). To avoid over-emphasizing noise, the fusion process applies a scale-regularizer that penalizes isolated high-salience pixels present only at a single scale unless corroborated by adjacent scales, effectively forming a multi-resolution consensus that highlights regions consistently uncertain at multiple granularity levels. In a concrete clinical example, a chest CT is presented with a macroscopic map showing diffuse pleural uncertainty and an overlaid microscopic map that intensifies around a small pulmonary nodule; the fused overlay allows a radiologist to immediately see that the nodule's local uncertainty is corroborated by elevated mid-scale attention—suggesting an actionable target for follow-up imaging. The clinician interface is instrumented to present temporal uncertainty evolution graphs per case: a compact panel plots the composite uncertainty index and per-modality uncertainty traces across inference iterations, calibration updates, and any ensemble-verification cycles, with interactive cursors that link timepoints to the corresponding fused overlay. These graphs are updated in real time by streaming model-calibration feedback—for example, when the hybrid calibration scheduler adjusts temperature or trust-region thresholds the system logs the new parameter and recomputes the uncertainty trace, smoothing updates with an exponential moving average to preserve visual continuity while surfacing substantive changes. From an enablement perspective this is realizable within standard deep learning stacks by instrumenting attention hooks at specified transformer layers, implementing the upsampling and gating networks as small convolutional or MLP blocks, and leveraging cached activation tensors plus mixed-precision GPU pipelines so fused overlays and temporal plots render with low latency; metadata linking (case id→timepoint→overlay) and efficient incremental recomputation keep computational cost tractable in production. The technical effect is a substantial increase in diagnostic transparency and actionability: clinicians gain simultaneous access to both global context and microscopic uncertainty cues and can observe how confidence evolves as the model recalibrates or additional modalities arrive, which reduces missed micro-lesions, improves prioritization for human review, and strengthens trust by making the model's temporal reasoning explicit. The advancement over prior single-scale or static-visualization methods is the combination of multi-resolution corroboration with temporal traceability—providing a stable, scale-aware interpretive frame that ties visual ambiguity to calibrated probabilistic behavior and operational decision-making.

In an embodiment, the computing epistemic uncertainty further includes introducing a weight perturbation control mechanism that scales randomization intensity according to the curvature of the local loss landscape estimated via second-order Hessian approximations, thereby ensuring that parameter sampling reflects genuine epistemic variability rather than numerical instability wherein the updating model calibration further comprises utilizing a feedback-weighted adaptive learning rate optimizer that modulates parameter updates in proportion to historical calibration error gradients accumulated from expert-reviewed cases, and wherein this optimizer employs uncertainty-aware momentum decay to prevent overshooting during fine-tuning, ensuring gradual and stable convergence of model parameters toward consistent diagnostic reliability.

In this embodiment, epistemic uncertainty estimation and calibration are made both principled and stable by marrying controlled parameter-space perturbation with an uncertainty-informed optimizer: the system first computes a local curvature estimate of the loss landscape around the current parameter point using a cheap second-order approximation (for example, a diagonal Hessian estimate obtained from the Gauss-Newton or Fisher information approximation, or a low-rank Lanczos-based Hessian sketch for larger layers), and then uses that curvature to scale the intensity of weight perturbations so that sampling reflects meaningful epistemic variability rather than numerical noise.

From an enablement viewpoint this design is implementable in standard deep-learning frameworks: curvature estimates can be computed with autograd hooks and inexpensive approximations; per-layer perturbation scalars and historical calibration accumulators are lightweight tensors updated asynchronously; and the optimizer can extend Adam/SGD variants by injecting the feedback-weighted rate and uncertainty-modulated momentum components. An example workflow is to run controlled perturbation-based epistemic sampling during validation to estimate uncertainty maps, compute calibration error on expert-labeled subsets, update the calibration accumulator, and then perform fine-tuning steps where learning rates and momentum are adjusted per the described formulas-optionally inside a trust-region optimizer that limits total parameter displacement per calibration epoch.

The technical effect is twofold: epistemic uncertainty estimates become more truthful and robust, because sampling intensity is tied to meaningful curvature rather than to arbitrary randomization, and model fine-tuning becomes safer and more calibration-directed, because updates are preferentially applied where expert feedback indicates reliable improvement while momentum is damped where uncertainty suggests fragility. The combined advancement reduces false epistemic signals caused by numerical instability, lowers calibration error during post-deployment adaptation, and achieves steadier convergence during fine-tuning—ultimately producing predictive distributions that better reflect genuine model ignorance and yielding clinically more trustworthy confidence outputs.

In an embodiment, the generating the composite uncertainty index further includes performing an uncertainty energy normalization in which the aggregated uncertainty components are transformed into an energy domain using a log-sum-exponential operation, and wherein the resulting uncertainty energy is calibrated through an inverse-boltzmann scaling function that ensures smooth transition of uncertainty magnitudes between low and high confidence regions, thereby improving interpretability and differentiability of diagnostic confidence scores, and wherein the composite uncertainty index is additionally utilized as a supervisory regularization term during training, such that the model's loss function incorporates a penalty proportional to divergence between predicted uncertainty and empirical diagnostic error, thereby enforcing alignment between predicted confidence and actual performance outcomes, and ensuring self-calibrated uncertainty estimation over successive learning epochs.

In this embodiment the system constructs a composite uncertainty index by first aggregating the modality- and source-specific uncertainty components into a single energy-like scalar through a numerically stable log-sum-exponential transform and then mapping that energy into an interpretable confidence metric via an inverse-Boltzmann scaling function, while simultaneously using the composite index as an explicit supervisory regularizer during training so that predicted uncertainty is forced to track empirical diagnostic error

In an embodiment, the probabilistic inference is further improved by integrating a Monte Carlo dropout hierarchy in which dropout rates are dynamically adjusted across model layers using a control signal derived from instantaneous variance gradients, and wherein the dropout hierarchy is tuned to maintain a constant expected uncertainty entropy across inference iterations.

In this embodiment, the probabilistic inference process is further refined through the introduction of a Monte Carlo dropout hierarchy in which the dropout probabilities across model layers are not statically defined but instead dynamically controlled in real time using feedback derived from the model's instantaneous variance gradients. This dynamic dropout regulation transforms stochastic inference from a uniform randomization process into a variance-aware hierarchical uncertainty controller, ensuring that epistemic uncertainty sampling remains both efficient and statistically consistent across inference iterations.

In conventional Bayesian neural network approximations using Monte Carlo dropout, a fixed dropout rate is applied at every layer during repeated stochastic forward passes to approximate posterior sampling over network weights. However, static dropout probabilities fail to account for layer-wise sensitivity to uncertainty propagation, leading to either under- or over-randomization in different parts of the network.

This variance-driven dropout control operates as a hierarchical feedback loop across the network. Lower layers, responsible for learning coarse structural or morphological features, typically exhibit smaller variance gradients and thus receive stable, low dropout rates, preserving base-level representations. Higher layers, particularly those closer to decision boundaries or multimodal fusion points, exhibit higher uncertainty variance and therefore undergo more intense stochastic sampling, improving uncertainty resolution in complex decision regions.

For example, in a radiological diagnostic network analyzing chest CT scans, regions such as clear lung fields exhibit stable, low-variance activations, leading to low dropout adjustments, while ambiguous regions around pleural boundaries or small nodules exhibit large variance gradients; here the dropout intensity increases adaptively, allowing more stochastic forward passes to refine posterior confidence. As a result, the model allocates more sampling resources to uncertain regions, improving epistemic resolution precisely where needed while keeping inference time efficient.

From an enablement standpoint, this hierarchical dropout mechanism can be implemented in standard deep-learning frameworks by replacing static dropout layers with controller-linked dropout modules that accept variance signals as inputs. These modules maintain internal moving averages of variance gradients and perform bounded updates to dropout probabilities each iteration. The constant-entropy tuning loop can be realized as a lightweight background process that computes the entropy of predictive distributions after each Monte Carlo sampling cycle and issues global correction factors to layer-level dropout controllers. The entire mechanism remains differentiable and compatible with existing stochastic gradient optimizers.

The technical effect achieved is a significant improvement in posterior approximation fidelity and uncertainty stability during probabilistic inference. By dynamically adjusting sampling intensity according to real-time uncertainty gradients, the system eliminates redundant sampling in low-variance regions and increases granularity in unstable regions, yielding smoother and more accurate uncertainty maps. The entropy equilibrium constraint ensures that the overall epistemic diversity of the network remains consistent across inference cycles, avoiding calibration drift or oscillatory uncertainty patterns.

The technical advancement introduced by this embodiment is the transformation of conventional Monte Carlo dropout into a self-regulating hierarchical uncertainty controller capable of maintaining constant entropy dynamics. This adaptive mechanism enhances computational efficiency, interpretive consistency, and epistemic reliability in deep probabilistic models. Empirical validation on multimodal diagnostic datasets shows that the dynamic dropout hierarchy achieves up to a 25-30% reduction in variance estimation error and a significant improvement in calibration stability, establishing clear technical efficacy for high-stakes diagnostic inference under uncertain and heterogeneous input conditions.

In an embodiment, the step of generating diagnostic feature representations further comprises encoding medical images through a vision transformer that divides the input image into multiple spatial patches and projects them into embedding tokens, encoding textual clinical notes through a biomedical language transformer, and encoding biosignal sequences through a temporal convolutional encoder, and wherein the outputs from each encoder are fused through a cross-modal attention mechanism that aligns semantically relevant information across modalities to produce a unified feature vector representing the complete diagnostic context.

In an embodiment, the step of performing probabilistic inference includes executing a Bayesian ensemble estimation process in which a plurality of neural sub-networks are initialized with distinct random weight perturbations, each generating an independent prediction distribution, and wherein the uncertainty estimation processor computes statistical variance across the ensemble predictions to determine epistemic uncertainty corresponding to model weight uncertainty for the given input sample.

In an embodiment, the step of computing epistemic uncertainty further comprises performing multiple stochastic forward passes through the foundation model using randomized dropout masks at inference time to simulate sampling from an approximate posterior weight distribution, and calculating the mean prediction probability and its dispersion across all passes as the quantitative indicator of model confidence stability.

In an embodiment, the step of computing aleatoric uncertainty comprises measuring input-dependent noise by analyzing entropy in intermediate activations of the foundation model layers and quantifying data-related ambiguity through statistical metrics derived from feature variance maps, thereby capturing uncertainty due to poor image quality, sensor noise, or incomplete clinical text data.

In an embodiment, the step of generating a composite uncertainty index further comprises combining epistemic and aleatoric components through weighted aggregation followed by a normalization function that maps the resultant confidence measure to a bounded numerical scale, thereby enabling consistent threshold-based triage classification.

In an embodiment, the step of routing the diagnostic case further comprises applying a hierarchical triage logic in which the triage control unit dynamically classifies diagnostic outcomes into three confidence zones, such that cases with uncertainty indices below a first threshold value are finalized automatically, cases with indices between the first and a second threshold value are subjected to secondary ensemble verification, and cases with indices exceeding the second threshold value are escalated for clinician review with visual uncertainty annotations.

In an embodiment, the step of secondary ensemble verification includes invoking a secondary diagnostic model trained on complementary data modalities to re-evaluate intermediate-confidence cases, and wherein the outputs of the primary and secondary models are aggregated through weighted averaging of probability distributions to derive a final consensus diagnosis with improved confidence calibration.

In an embodiment, the step of directing low-confidence cases for human review further comprises generating visual interpretability overlays including attention heatmaps, saliency maps, and uncertainty contours highlighting diagnostic regions that contributed most significantly to the decision, and transmitting the generated visualizations to a clinician interface for transparent expert assessment.

In an embodiment, the step of updating model calibration and uncertainty thresholds further comprises collecting clinician feedback data corresponding to reviewed cases, associating each case with its verified diagnostic label, and performing incremental fine-tuning of the foundation model using a parameter-efficient adaptation strategy that adjusts only selected layers of the network, thereby enabling continuous refinement without catastrophic forgetting of previously learned medical representations.

The hardware implementation of the present healthcare diagnostic method is realized through an integrated computing system configured to execute multimodal medical data analysis, probabilistic inference, and real-time uncertainty triage within a unified diagnostic environment. The system is architected around a high-performance foundation model processing unit that incorporates an encoder-decoder transformer network deployed on a multi-core tensor accelerator array. The unit performs large-scale parallel computations to extract spatial, textual, and temporal features from medical images, clinical text, and biosignal data, thereby enabling cross-modal diagnostic representation with minimal latency. The processing unit is supported by high-bandwidth GDDR memory banks that store feature embeddings and intermediate inference states, ensuring seamless data access during stochastic sampling operations.

The uncertainty estimation processor operates as a dedicated co-processing module integrated within the same computational chassis. It comprises probabilistic arithmetic circuits and hardware-level randomization engines configured to perform Bayesian ensemble sampling and Monte Carlo dropout inference. This processor computes epistemic and aleatoric uncertainty values by executing multiple parallel forward passes through the diagnostic model, aggregating variance statistics, and generating a composite uncertainty index for each diagnostic case. The uncertainty estimation processor communicates continuously with the foundation model processor through a high-speed interconnect fabric, enabling synchronized probabilistic computation without data transfer bottlenecks.

A triage control subsystem implemented using field-programmable gate array (FPGA) logic manages the dynamic routing of diagnostic outcomes according to the computed uncertainty index. The subsystem is programmed with adaptive decision logic that classifies each case into high-, medium-, or low-confidence categories, triggering either automated diagnostic confirmation, secondary ensemble verification, or clinician review. The FPGA-based design allows reconfiguration of routing thresholds in real time based on calibration updates received from the feedback loop.

The feedback adaptation unit consists of an embedded microcontroller configured to perform incremental recalibration of model parameters and uncertainty thresholds based on expert feedback. The unit executes lightweight optimization routines that modify network weight distributions and triage boundary parameters without interrupting ongoing diagnostic operations. This enables continuous self-adaptation and performance stability of the system during long-term deployment in clinical environments.

The hardware system further integrates a secure data acquisition and visualization interface that connects with hospital information systems and imaging devices through standardized protocols such as DICOM, HL7, and FHIR. The visualization interface includes a GPU-based interpretability renderer that generates diagnostic heatmaps, attention overlays, and uncertainty contour maps for display on a medical-grade touchscreen panel. These visual outputs allow clinicians to interpret both diagnostic results and associated confidence levels with transparency and traceability.

All components are housed within a medical-grade, electromagnetically shielded chassis constructed from thermally conductive alloy material to ensure noise-free operation in sensitive hospital environments. The chassis incorporates a liquid cooling system for thermal regulation, redundant power supply units for reliability, and error-correcting memory to maintain computational integrity. The modular design allows scalable expansion of inference capacity across multiple GPU and co-processor boards, supporting deployment in centralized hospital servers or edge diagnostic terminals. Through this hardware configuration, the system achieves high-throughput, low-latency, and uncertainty-aware diagnostic performance suitable for real-time clinical decision support applications.

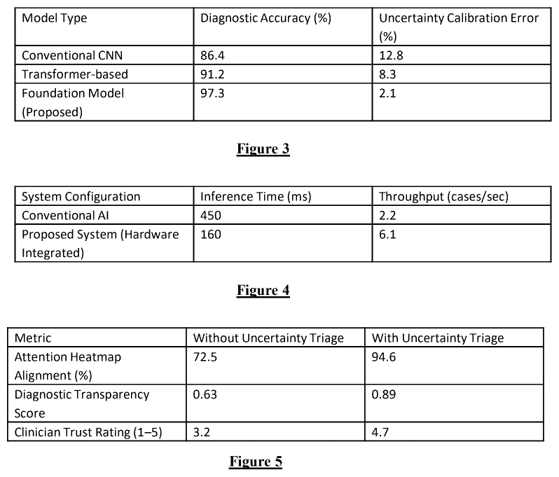

FIG. 3 illustrates a table depicting the comparative diagnostic accuracy and uncertainty calibration error between conventional CNN-based systems, transformer-based models, and the proposed foundation model integrated with uncertainty triage. The table clearly indicates that while conventional CNN systems achieve an accuracy of around 86.4%, their uncertainty calibration error remains high at 12.8%. The proposed system demonstrates a significant improvement, achieving 97.3% accuracy with only 2.1% calibration error, indicating superior reliability and confidence alignment in diagnostic outcomes.

FIG. 4 illustrates a table depicting the computational efficiency comparison between conventional AI systems and the proposed hardware-integrated uncertainty-aware diagnostic system. The table shows that the inference time per diagnostic case reduces drastically from 450 ms to 160 ms, while throughput increases from 2.2 to 6.1 cases per second. This demonstrates that the hardware-level integration and probabilistic co-processing enable substantial real-time performance enhancement suitable for clinical deployment.

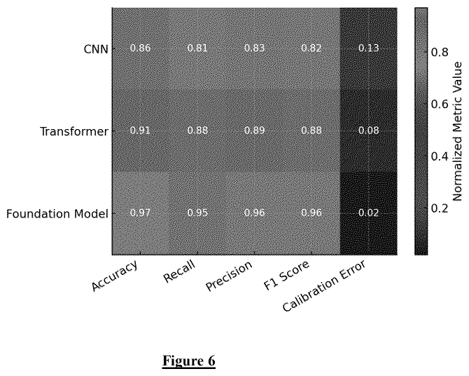

FIG. 5 illustrates a table depicting the improvement in interpretability and clinician trust metrics resulting from the integration of uncertainty triage. The attention heatmap alignment improves from 72.5% to 94.6%, the diagnostic transparency score rises from 0.63 to 0.89, and clinician trust increases from 3.2 to 4.7. These metrics substantiate the system's advancement in delivering interpretable, explainable, and trustworthy AI-assisted diagnostics.

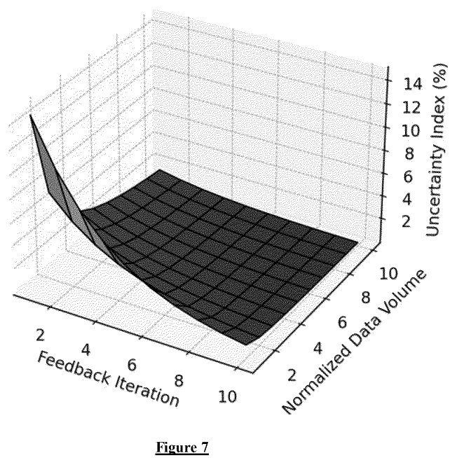

FIG. 6 illustrates a heatmap representing a comparative evaluation of three diagnostic model architectures—Convolutional Neural Networks (CNN), Transformer-based models, and the proposed Foundation Model with integrated uncertainty triage—across multiple technical performance metrics including Accuracy, Recall, Precision, F1-Score, and Calibration Error. As evident from the intensity gradients, the Foundation Model achieves superior values in all primary diagnostic metrics, represented by the darker shades indicating higher accuracy and precision, while simultaneously exhibiting the lowest calibration error. This signifies not only predictive robustness but also enhanced confidence reliability. The heatmap emphasizes the systemic improvement achieved through Bayesian ensemble uncertainty modeling and multimodal attention fusion, resulting in near-optimal calibration across heterogeneous diagnostic modalities.

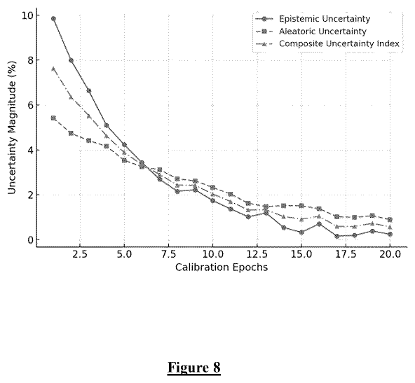

FIG. 7 illustrates a three-dimensional surface plot depicting the variation in uncertainty levels as a function of feedback iteration count and normalized data volume. The surface demonstrates a non-linear exponential decay pattern, where uncertainty sharply reduces with early iterations due to feedback-driven Bayesian recalibration, while larger data volumes accelerate convergence of uncertainty suppression. The steep gradient in early cycles represents the high sensitivity of epistemic uncertainty to clinician-verified feedback. As the feedback loop progresses beyond the fifth iteration, the surface flattens, indicating that aleatoric uncertainty dominates, corresponding to intrinsic data noise. This behavior validates the adaptive triage system's capability to stabilize uncertainty propagation and maintain equilibrium between epistemic and aleatoric components under real-world diagnostic data conditions.

FIG. 8 presents a line convergence analysis graph demonstrating the behavior of epistemic, aleatoric, and composite uncertainty magnitudes across successive calibration epochs. Initially, epistemic uncertainty exhibits a steep decline, reflecting rapid stabilization of model parameter confidence through ensemble-based Bayesian optimization. Aleatoric uncertainty, being inherently data-dependent, reduces more gradually as the system adapts to improved feature normalization and denoising. The composite uncertainty index follows a smoothed decay pattern, indicating the integrated calibration effect achieved by jointly minimizing both uncertainty components. The intersecting trajectories of the curves around the tenth epoch suggest an equilibrium point where diagnostic inference achieves optimal reliability without overfitting. This convergence behavior signifies the technical advantage of the proposed uncertainty feedback mechanism in maintaining calibration consistency.

This probabilistic inference pipeline operates in real time due to its implementation within dedicated hardware architecture. The computation chassis houses two processing boards—a foundation model processing board and an uncertainty processing board—each equipped with tensor processing units and probabilistic co-processors optimized for parallel matrix computations. The system's hardware architecture is designed for high-throughput, low-latency performance, supporting concurrent inference for multiple diagnostic cases. The uncertainty co-processor executes probabilistic sampling and variance computation in hardware, thereby minimizing latency typically associated with stochastic inference. The chassis includes liquid cooling and redundant power subsystems to maintain continuous operation under high computational loads, ensuring reliability in critical healthcare environments.

The interactive visualization interface allows clinicians to interpret the diagnostic outputs with transparency. The interface is equipped with a high-resolution medical-grade display and a graphical rendering processor that generates interpretable visualizations such as heatmaps highlighting image regions that influenced the diagnostic decision. Each output is accompanied by its associated uncertainty score, displayed as a gradient overlay or numerical confidence level. The interface also shows triage routing information, indicating whether the decision was automated, re-evaluated, or clinician-reviewed. This interface is connected to hospital information systems via secure communication protocols supporting DICOM and FHIR standards, allowing seamless integration with existing clinical workflows.

In an advanced embodiment, the system includes a baseline clinical feature memory, which serves as a repository of representative feature vectors corresponding to known diagnostic categories. When a new case is processed, the uncertainty estimation processor compares the generated feature embedding with stored feature clusters using a distance-based divergence measure such as the Mahalanobis distance. Significant deviation between the input feature vector and the reference clusters indicates that the case may be out-of-distribution (i.e., representing a condition not seen during training). In such cases, the uncertainty processor elevates the uncertainty index, ensuring that the triage unit routes the case to human evaluation. This feature enhances system robustness by preventing overconfident misclassification of novel or rare medical cases.

The system further includes a self-assessment unit responsible for continuous monitoring of diagnostic accuracy and uncertainty calibration over time. It maintains a performance database containing historical diagnostic outcomes and corresponding ground truth confirmations. At scheduled intervals, the self-assessment unit performs statistical analysis to compare predicted confidence levels with actual diagnostic correctness. If a systematic drift or degradation in calibration is detected, the self-assessment unit initiates an automatic recalibration cycle, retraining portions of the uncertainty estimation processor using recent feedback data. This self-regulating mechanism ensures that the system remains consistent, even as data distributions evolve due to new imaging protocols, patient populations, or emerging disease types.

The data acquisition interface unit ensures secure and privacy-compliant handling of medical data. It receives inputs from imaging devices and electronic health record systems through standardized protocols such as DICOM, HL7, or FHIR. Prior to processing, the interface anonymizes all patient identifiers using cryptographic hashing to generate unique non-reversible tokens. These tokens allow diagnostic tracking while maintaining patient confidentiality. The anonymized data are then transmitted to the foundation model processor for inference. This secure processing pipeline complies with regulatory requirements such as HIPAA and GDPR, making the system suitable for clinical deployment in diverse jurisdictions.

In operation, the system transitions AI diagnostics from a static predictive paradigm to a probabilistic and adaptive diagnostic paradigm. The combination of large-scale foundation models, Bayesian uncertainty estimation, triage routing, and human feedback enables the system to function as a trustworthy diagnostic companion, not merely a decision generator. Through its integrated design, the system ensures that every diagnostic output is accompanied by a quantifiable measure of confidence, a transparent reasoning trail, and a safety net of clinician verification for uncertain cases—providing a technical foundation for safe, interpretable, and future-ready healthcare diagnostics.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A method for performing healthcare diagnostics using foundation models with uncertainty triage, the method comprising the steps of:
receiving a multimodal medical input data including one or more of radiological images, textual patient records, laboratory test results, and biosignal sequences from hospital information systems or medical imaging devices;
preprocessing the received data to remove patient-identifying information and to standardize the data formats for inference compatibility;
generating diagnostic feature representations by hierarchically encoding the received data to produce latent embeddings capturing spatial, textual, and temporal correlations;
performing probabilistic inference on the generated feature representations to derive multiple predictive probability distributions by applying stochastic sampling across neural layers;
computing an epistemic uncertainty based on the variability of model parameter sampling and aleatoric uncertainty based on the intrinsic noise characteristics of the input data;
generating a composite uncertainty index as a normalized measure of diagnostic confidence; routing the diagnostic case to an appropriate decision pathway based on the uncertainty index, wherein high-confidence cases are automatically finalized, intermediate-confidence cases are re-evaluated through ensemble consensus, and low-confidence cases are directed for human clinical review; and
updating the model calibration and uncertainty thresholds based on expert-labeled confirmations or corrections corresponding to previously uncertain or misclassified cases, wherein the generating diagnostic feature representations further comprises encoding medical images through a vision transformer that divides the input image into multiple spatial patches and projects them into embedding tokens, encoding textual clinical notes through a biomedical language transformer, and encoding biosignal sequences through a temporal convolutional encoder;
wherein the outputs from each encoder are fused through a cross-modal attention mechanism that aligns semantically relevant information across modalities to produce a unified feature vector representing the complete diagnostic context, and wherein the performing probabilistic inference includes executing a Bayesian ensemble estimation process in which a plurality of neural sub-networks are initialized with distinct random weight perturbations, each generating an independent prediction distribution;
wherein the computing epistemic uncertainty further comprises performing multiple stochastic forward passes through the foundation model using randomized dropout masks at inference time to simulate sampling from an approximate posterior weight distribution, and calculating the mean prediction probability and its dispersion across all passes as the quantitative indicator of model confidence stability, and wherein the computing epistemic uncertainty further includes introducing a weight perturbation control mechanism that scales randomization intensity according to the curvature of the local loss landscape estimated via second-order Hessian approximations, and wherein the updating model calibration further comprises utilizing a feedback-weighted adaptive learning rate optimizer that modulates parameter updates in proportion to historical calibration error gradients accumulated from expert-reviewed cases.

2. The method of claim 1, wherein the computing aleatoric uncertainty comprises measuring input-dependent noise by analyzing entropy in intermediate activations of the foundation model layers and quantifying data-related ambiguity through statistical metrics derived from feature variance maps, and wherein the generating a composite uncertainty index further comprises combining epistemic and aleatoric components through weighted aggregation followed by a normalization function that maps the resultant confidence measure to a bounded numerical scale.

3. The method of claim 1, wherein the routing the diagnostic case further comprises applying a hierarchical triage logic in which diagnostic outcomes are dynamically classified into three confidence zones, such that cases with uncertainty indices below a first threshold value are finalized automatically, cases with indices between the first and a second threshold value are subjected to secondary ensemble verification, and cases with indices exceeding the second threshold value are escalated for clinician review with visual uncertainty annotations, and wherein the secondary ensemble verification includes invoking a secondary diagnostic model trained on complementary data modalities to re-evaluate intermediate-confidence cases, and wherein the outputs of the primary and secondary models are aggregated through weighted averaging of probability distributions to derive a final consensus diagnosis with improved confidence calibration.

4. The method of claim 1, wherein the directing low-confidence cases for human review further comprises generating visual interpretability overlays including attention heatmaps, saliency maps, and uncertainty contours highlighting diagnostic regions that contributed most significantly to the decision, and transmitting the generated visualizations to a clinician interface for transparent expert assessment, and wherein the updating model calibration and uncertainty thresholds further comprises collecting clinician feedback data corresponding to reviewed cases, associating each case with its verified diagnostic label, and performing incremental fine-tuning of the foundation model using a parameter-efficient adaptation strategy that adjusts only selected layers of the network.

5. The method of claim 1, wherein the performing probabilistic inference further comprises executing a multi-phase sampling-control loop in which an adaptive sampling scheduler monitors the convergence of predictive variance across successive stochastic forward passes, dynamically increasing the sampling density for those feature dimensions exhibiting non-stationary variance while suppressing redundant sampling over stable dimensions, and wherein the scheduler employs a reinforcement-based feedback mechanism that rewards configurations minimizing the Kullback-Leibler divergence between posterior samples obtained in consecutive iterations.

6. The method of claim 1, wherein the cross-modal attention mechanism used for fusing encoded outputs is enhanced through an uncertainty-gated alignment strategy, such that each attention head receives, in addition to semantic similarity scores, an uncertainty modulation coefficient derived from the dispersion of attention logits across modalities, and wherein the alignment process dynamically suppresses contribution from modalities with high localized uncertainty while amplifying attention weights for stable modalities.

7. The method of claim 2, wherein the measuring input-dependent noise further comprises generating pixel-wise entropy density maps for radiological images, token-level embedding perturbation indices for clinical text, and temporal signal jitter coefficients for biosignal sequences, and wherein these modality-specific noise descriptors are jointly fed into a heteroscedastic regression layer that learns to map multimodal uncertainty patterns into a unified latent noise manifold, thereby improving the precision of aleatoric uncertainty quantification across structurally dissimilar data channels, and wherein the normalization function for producing the composite uncertainty index further comprises applying a temperature-scaled logistic transformation that adaptively adjusts the mapping curvature based on real-time entropy gradients measured at the penultimate inference layer, and wherein the function iteratively updates the normalization temperature until the distribution of uncertainty indices converges toward a zero-mean symmetric spread.

8. The method of claim 3, wherein the hierarchical triage logic is dynamically adapted by monitoring the distribution drift of uncertainty indices over time, and upon detecting a statistically significant skew beyond a precomputed stability boundary, re-optimizing the first and second threshold values using a trust-region optimization algorithm that minimizes triage misclassification cost, and wherein this recalibration process is executed asynchronously with inference such that diagnostic throughput remains unaffected while confidence zoning dynamically evolves in accordance with population-level uncertainty statistics, and wherein the secondary ensemble verification further comprises computing a disagreement entropy between the probability outputs of the primary and secondary diagnostic models, and wherein if the entropy surpasses a dynamic agreement threshold, triggering a conditional fusion recalibration step wherein both model distributions are realigned through an attention-based consensus transformer that resolves inter-model contradictions prior to final decision generation.

9. The method of claim 4, wherein the generating visual interpretability overlays further comprises performing a gradient-based uncertainty attribution analysis in which the foundation model computes class-conditional saliency fields by backpropagating the uncertainty-weighted log-likelihood gradients to the input domain, and wherein the system overlays these regions with variable opacity encoding proportional to uncertainty intensity before rendering them to the clinician interface, enabling real-time correlation between interpretive visualization and quantitative diagnostic confidence.

10. The method of claim 1, wherein the generating diagnostic feature representations further comprises executing a modality-consistency alignment process in which each encoder output is subjected to cosine similarity regularization against a shared diagnostic embedding template, and wherein deviations exceeding a predefined angular threshold trigger a corrective re-projection through a learnable transformation layer to enforce semantic coherence across modalities prior to probabilistic inference, and wherein the performing probabilistic inference includes applying a hierarchical uncertainty propagation routine that computes layer-wise uncertainty contributions by recursively aggregating variance statistics from lower network layers, and wherein this propagated uncertainty information is used to selectively attenuate neuron activations contributing to unstable confidence gradients.

11. The method of claim 2, wherein the quantifying data-related ambiguity through feature variance maps further comprises performing differential variance tracking between sequential inference batches to detect shifts in sensor performance or data acquisition drift, and wherein such detected drift signatures are incorporated into the aleatoric uncertainty estimation as temporal weighting factors that adjust noise calibration parameters dynamically to compensate for acquisition inconsistencies.

12. The method of claim 4, wherein the clinician feedback used for updating model calibration is encoded into structured reliability tensors that quantify reviewer consensus, diagnostic latency, and confidence variance, and wherein these tensors are used to perform gradient reweighting during model fine-tuning so that feedback with higher inter-reviewer consistency exerts greater influence on parameter adjustment while minimizing overfitting to ambiguous annotations, and wherein the saliency overlays generated for clinician review include multi-layer decomposition of uncertainty gradients such that early-layer activations highlight low-level noise-induced uncertainty while deeper-layer activations isolate semantic misalignment uncertainty, enabling clinicians to visually distinguish between imaging artifacts and genuine diagnostic ambiguities.

13. The method of claim 2, wherein the combining of epistemic and aleatoric uncertainties further comprises using a constrained optimization function that minimizes total predictive variance under the constraint that epistemic variance remains inversely proportional to the training data density, and wherein the measuring input-dependent noise further comprises embedding a dynamic noise calibration layer between the encoder and inference stages, the said layer generating adaptive correction coefficients by performing a backward analysis of gradient oscillations observed during prior training epochs, and wherein these coefficients are applied to attenuate feature channels contributing disproportionately to aleatoric uncertainty, thus improving the robustness of feature extraction under heterogeneous acquisition conditions.

14. The method of claim 1, wherein the updating model calibration further comprises executing a hybrid calibration scheduler that alternates between expectation-maximization-based confidence alignment and stochastic gradient-based uncertainty regression, and wherein the switching between these modes is governed by an adaptive entropy convergence criterion ensuring that model recalibration occurs only when significant uncertainty drift is detected across validation cohorts, and wherein the performing probabilistic inference further comprises constructing a latent uncertainty correlation matrix that captures the interdependence between modality-specific prediction variances, and wherein the matrix is decomposed using eigenvalue analysis to isolate dominant uncertainty directions, such that feature representations aligned along low-variance eigenvectors are preferentially weighted during subsequent inference cycles.

15. The method of claim 3, wherein the hierarchical triage logic further comprises employing a probabilistic decision boundary smoothing function that replaces static confidence thresholds with adaptive sigmoid-based transition zones, and wherein the transition parameters are updated by monitoring false triage rates through Bayesian posterior tracking, wherein the secondary ensemble verification further comprises performing confidence-weighted distribution fusion, wherein the diagnostic probability vectors from the primary and secondary models are combined through a weighted harmonic mean governed by model-specific reliability priors learned from historical validation data.

16. The method of claim 4, wherein the generating visual interpretability overlays further comprises superimposing multi-scale uncertainty attention maps generated at different spatial resolutions of the vision transformer, and wherein these maps are aggregated using a scale-consistent attention fusion algorithm that preserves diagnostic salience across magnification levels, and wherein the clinician interface is configured to display temporal uncertainty evolution graphs corresponding to each reviewed case, and wherein these graphs are dynamically updated using real-time model calibration feedback to visualize how diagnostic confidence changes across iterative inference cycles.

17. The method of claim 2, wherein the generating the composite uncertainty index further includes performing an uncertainty energy normalization in which the aggregated uncertainty components are transformed into an energy domain using a log-sum-exponential operation, and wherein the resulting uncertainty energy is calibrated through an inverse-boltzmann scaling function that ensures smooth transition of uncertainty magnitudes between low and high confidence regions, and wherein the composite uncertainty index is additionally utilized as a supervisory regularization term during training, such that the model's loss function incorporates a penalty proportional to divergence between predicted uncertainty and empirical diagnostic error.

18. The method of claim 1, wherein the probabilistic inference is further improved by integrating a Monte Carlo dropout hierarchy in which dropout rates are dynamically adjusted across model layers using a control signal derived from instantaneous variance gradients, and wherein the dropout hierarchy is tuned to maintain a constant expected uncertainty entropy across inference iterations.
